(19) United States
(12) Patent Application Publication
(10) Pub. No.: US 2023/0204588 A1
(43) Pub. Date: Jun. 29, 2023

(54) Method for Enriching Extracellular Vesicles From Biological Fluid Samples

(71) Applicant: Genentech Inc., South San Francisco, CA (US)

(72) Inventors: Christopher J. Lowe, Moraga, CA (US); Vicki Plaks, Santa Monica, CA (US); Jane M. Ruppel, Paso Robles, CA (US); Rebecca Suttmann, Menlo Park, CA (US)

(73) Assignee: Genentech Inc., South San Francisco, CA (US)

(21) Appl. No.: 17/965,363

(22) Filed: Oct. 13, 2022

Related U.S. Application Data
(63) Continuation of application No. PCT/US2021/027627, filed on Apr. 16, 2021.
(60) Provisional application No. 63/011,583, filed on Apr. 17, 2020.

(51) Int. Cl. G01N 33/574; G01N 33/543

(52) U.S. Cl. CPC G01N 33/57484; G01N 33/54326; G01N 2333/70596

(57) ABSTRACT

The invention provides methods for enriching extracellular vesicles (EVs), including exosomes, from biological fluid samples from subjects, and optionally further testing the EVs for the presence of specific biomarkers.

### FIELD

The invention provides methods for enriching extracellular vesicles (EVs), including exosomes, from biological fluid samples from subjects, and optionally further testing the EVs for the presence of specific biomarkers.

### BACKGROUND

Extracellular vesicles (EVs) are cellular particles shed from cells that may be found in various bodily fluids. EVs include exosomes (which can be about 40 to about 100 nm), microvesicles (which can be about 100 nm to about 1 µm) and apoptotic bodies (which can be about 1 to about 5 µm), for example. These EVs have been shown to play an important role in intercellular communication in homeostatic and oncogenic processes (reviewed in Becker et al., 2016). Exosomes arise from intraluminal vesicles, which are byproducts of the endocytic process and exist in the cytoplasm. Once these precursors are secreted from the plasma membrane of cells, they become exosomes. Microvesicles (sometimes called ectosomes) are assembled directly at the plasma membrane and may contain similar cargo and surface markers as exosomes (such as tetraspanins).

EVs are heterogeneous as they are secreted from various tissues, and even those secreted from a single site can be heterogeneous, depending on their cell of origin. Databases such as EVpedia® (www.evpedia.info) and ExoCarta® (www.exocarta.org) exist to catalog the extensive repertoire of molecules found within EVs from various sources (Mathivanan and Simpson, 2009; Kim et al. 2015). However, there is considerable debate as to what is the best technique to isolate EVs (including exosomes and microvesicles). The great majority of literature is focused on exosomes, with microvesicles less-studied. Furthermore, the technique utilized for EV isolation often depends on the downstream application. Differential ultracentrifugation (DUC), where successive centrifugation steps are employed with increasing speed and time, is one of the most commonly used methods (Andre et al. 2002; Pisitkun et al. 2004). Sequential filtration, with or without ultracentrifugation, can be used to ensure a size-specific fraction of EVs. Additionally, commercial reagents like ExoQuick® (System Biosciences) and Total Exosome Isolation (Thermo Fisher) are capable of isolating these particles. However, neither of these methods addresses how to enrich for EVs that originate from a certain cell type, for example, such as blood-circulating EVs from particular cell types.

The present disclosure provides, inter alia, novel methods of enriching EVs from biological fluid samples from mammalian subjects based on presence of cell surface markers, and for optionally further testing the isolated EVs for the presence of additional biomarkers, such as biomarkers that may be found either on the surface or in the lumen of EVs and that may be associated with particular cell types or disease states. EVs shed from tumors, for example, contain tumor-derived proteins and nucleic acids (Rabinowits et al., 2009; Inal et al. 2013). The ability to specifically enrich for these will enable the study of tumor-specific information in liquid biopsy over time and across multiple metastatic foci.

### SUMMARY

This disclosure, in some embodiments, relates to processes of enriching extracellular vesicles (EVs) in a biological fluid sample from a subject. In some embodiments, the EVs are cell type-specific. In some embodiments, the processes comprise (a) providing a biological fluid sample from the subject; (b) contacting the sample with antibodies specific for a cell surface marker for one or more cell types, optionally wherein the antibodies are attached to a matrix; and (c) isolating antibody-bound EVs from unbound EVs the sample. In some embodiments, the sample comprises a volume from 100 µl to 7 mL, from 200 µl to 6.5 mL, from 250 µl to 6.5 mL, from 200 µl to 6 mL, from 250 µl to 6 mL, from 200 µl to 5 mL, from 200 µl to 4 mL, from 200 µl to 3 mL, from 200 µl to 2 mL, from 100 µl to 1 mL, from 150 µl to 1 mL, from 200 µl to 1 mL, from 250 µl to 1 mL, from 200 µl to 500 µl, from 200 µl to 500 µl, from 200 µl to 400 µl, from 250 µl to 500 µl, or from 250 µl to 400 µl. In any of the above processes, the EVs may comprise EVs from epithelial cells, lung tissue cells, breast tissue cells, liver tissue cells, prostate tissue cells, kidney tissue cells, urinary tract cells, neural cells, tumor cells, solid tumor cells, lung tumor cells, breast tumor cells, liver tumor cells, prostate tumor cells, kidney tumor cells, urinary tumor cells, glioblastoma cells, or amyloid-beta expressing cells. In some embodiments, the cell surface marker is an epithelial cell surface marker. In some embodiments, the cell surface markers may be (a) an epithelial cell surface marker, such as EpCAM, EGFR, PSMA, GSP64, CD3, CD49b, CD87, CD95, E-Cadherin CA9, CA12, N-cadherin, OB-cadherin, cadherin-11, a cytokeratin or epithelial membrane antigen (EMA); (b) a lung tissue marker, such as EpCAM, CA9, CA12, DSG3, FAT2, GPR87, KISS1R, LYPD3, SLC7A11, TMPRSS4, CD133, prominin-1, AC133, programmed death-1 receptor (PD1) or FAS; (c) a breast tissue marker, such as E-cadherin, epithelial membrane antigen (EMA), human epidermal growth factor receptor type 2 (Her2/neu), αvβ6 integrin, EpCAM, carcinoembryonic antigen (CEA), folate receptor-alpha (FR-α), urokinase-type plasminogen activator receptor (uPAR) or placental-specific protein 1 (PLAC1); (d) a liver cell marker, such as CD133, Prominin-1, CD44, EpCAM, delta-like 1 non-canonical Notch ligand 1 (DLK1), ALDH, CD13, CD90, CD24, OV6, ICAM-1, CD34, C-kit, α2δ1, K19, LGR5, GPC3, Annexin A2, CD15, ABC transporters, Nope, DCLK1, ASGPR, CK or CD47; (e) a glioblastoma biomarker, such as fibronectin, CD63, HSP70, Annexin A2, CD9, CD81, CD44, GRP78, CD133, CD15, a sialoglycoprotein, SLC1A3, PTPRZ1, GPR56, CLU or ALD1A3; (f) a urinary tract cell marker, such as a tetraspanin, CD9, CD81, LAMP-1, CD10, CD24, CD44 or CD63; or (g) an amyloid marker such as beta-amyloid precursor protein (β-APP), presenilin protein PS-1, or proinsulin protein PS-2. In some embodiments, the cell surface marker can be EpCAM.

In some embodiments, wherein the antibodies are attached to a matrix, the matrix may comprise particles such as beads, pellets, or chips, which are optionally magnetic. In some embodiments, the biological fluid sample may comprise tears, saliva, lymph fluid, urine, serum, cerebral spinal fluid, pleural effusion, ascites, or plasma. In some embodiments, the sample comprises serum or plasma.

In any of the above processes, the subject may be a human. In some embodiments, the subject may be a cancer subject or suspected cancer subject. In some embodiments, the cancer is a solid tumor. In some embodiments, the cancer is breast cancer, triple negative breast cancer, lung cancer, NSCLC, SCLC, liver cancer, urinary tract cancer, bladder cancer, brain cancer, or glioblastoma. In some embodiments, the subject may be an infectious disease subject or suspected infectious disease subject. In some embodiments, the subject may be an inflammatory disease subject or suspected inflammatory disease subject. In some embodiments, where the subject is a cancer subject or suspected cancer subject, the cell surface marker is EpCAM.

In some embodiments, the subject may be an amyloid disease subject or suspected amyloid disease subject.

In some cases, the methods may further comprise isolating total extracellular vesicles (EVs) from the sample, such as by membrane capture or differential ultracentrifugation, determining total protein levels in the isolated total extracellular vesicles, and optionally comparing the total protein levels from the total EVs to levels of cell surface marker in the EVs.

In some embodiments, isolation of antibody bound EVs from unbound EVs is through attachment of antibodies to a matrix. In some embodiments, isolation of the antibody bound EVs from unbound EVs comprises separating the matrix from supernatant in the sample, such as by allowing the matrix to precipitate by gravity or centrifugation.

In some embodiments, the processes may include contacting the antibody bound EVs with a detection agent specific for a second biomarker. In some embodiments, the antibody bound EVs are resuspended in lysis buffer prior to being contacted with the detection agent specific for the second biomarker. In some embodiments, the detection agent is an antibody. In some embodiments, the second biomarker is a protein, a polynucleotide (e.g. an RNA molecule), a lipid, a drug, or a drug metabolite. In some embodiments, the subject has cancer or is suspected of having cancer, and where the second biomarker is a polypeptide expressed or overexpressed in tumor cells, and/or wherein the second biomarker is a cytokine, cytokine receptor, or an immune checkpoint regulator. In some embodiments, the second biomarker is one or more of PD-L1, SCF, IL-3, GM-CSF, G-CSF, M-CSF, TNF-alpha, IL-2, IL-5, TMB, CTLA-4, ICOS, 4-1BB (CD137), PD-1, CTLA-4, LAG-3, Tim-3, CD39, IFN-β, IFN-y, IL-2, IL-10, TGF-β, CCR10, CXCR4, CCR7, sMICA, IL-8, IDO1, GBP1, class II MHC molecules, CXCL9, CXCL10 (IP-10), CXCL11, IL-6, CCL4, CCL5, IFNGR1, IFNGR2, JAK2, IRF1, IFIT1, IFIT2, MTAP, miR3, SOCA1, PIAS4, GZMA, GZMB, PRF1, HLA-DQA1, HLA-DRB1, IFNG, STAT1, ICAM1-5, VCAM-1, JAK1, JAK2, CCR5, HLA-DRA, CXCR6, TIGIT, CD27, CD274, PDCD1LG2 (PD-L2), LAG3, NKG7, PSMB10, CMKLR1, CD8A, CD8B, HLA-DB1, HLA-E, CD276, CD3D, CD3E, CD3G, CD247, ZAP70, CD2, CD28, ICOS, IL12Rb1, GZMM, FLTSLG, IL-15, Eotazin, GRO-1, VEGF, HGF, AFP, BCR-ABL, BRCA1/BRCA2, B-Raf V600E, CA-125, CA19-9, CEA, EGFR, HER-2/neu, KIT, PSA, S100, KRAS, UGT1A1 or CD20.

The processes described above may also have one or more of the following characteristics: (a) the sample is not subjected to chromatography either before or after contacting the sample with the antibodies; (b) the sample is not treated before contacting the sample with the antibodies other than optionally to remove particles above 1 µm, above 2 µm, above 5 µm, above 10 µm, or above 20 µm in diameter and/or to remove cellular bodies and debris larger than EVs; (c) the EVs are not subjected to chromatography either before or after contacting the sample with the antibodies; (d) the antibodies are attached to a matrix, and the matrix is not a filter, an ion-exchange medium, or a membrane; or (e) the antibodies are attached to a matrix and the matrix is not charged.

In some processes herein, isolation of cell-type specific EVs from the sample consists essentially of (a) contacting the sample with antibodies specific for the cell surface marker for the one or more cell types (optionally wherein the antibodies are attached to the matrix), and (b) isolating the antibody-bound EVs from unbound EVs the sample.

This disclosure, in some embodiments, also relates to processes of enriching cell-type specific extracellular vesicles (EVs) from a subject, comprising: (a) providing a biological fluid sample from the subject; (b) contacting the sample with antibodies specific for a cell surface marker, optionally wherein the antibodies are attached to a matrix; (c) isolating antibody-bound EVs from unbound EVs the sample; (d) optionally resuspending the antibody-bound EVs in a lysis buffer; and (e) contacting the EVs with a detection agent specific for a second biomarker. In some embodiments, the sample comprises a volume from 100 µl to 7 mL, from 200 µl to 6.5 mL, from 250 µl to 6.5 mL, from 200 µl to 6 mL, from 250 µl to 6 mL, from 200 µl to 5 mL, from 200 µl to 4 mL, from 200 µl to 3 mL, from 200 µl to 2 mL, from 100 µl to 1 mL, from 150 µl to 1 mL, from 200 µl to 1 mL, from 250 µl to 1 mL, from 200 µl to 500 µl, from 200 µl to 500 µl, from 200 µl to 400 µl, from 250 µl to 500 µl, or from 250 µl to 400 µl. In some of these processes, the EVs may be EVs from epithelial cells, lung tissue cells, breast tissue cells, liver tissue cells, prostate tissue cells, kidney tissue cells, urinary tract cells, neural cells, tumor cells, solid tumor cells, lung tumor cells, breast tumor cells, liver tumor cells, prostate tumor cells, kidney tumor cells, urinary tumor cells, glioblastoma cells, or amyloid-beta expressing cells. In some embodiments, the cell surface marker is an epithelial cell surface marker.

In some of these processes, the cell surface marker is: (a) an epithelial cell surface marker, such as EpCAM, EGFR, PSMA, GSP64, CD3, CD49b, CD87, CD95, E-Cadherin CA9, CA12, N-cadherin, OB-cadherin, cadherin-11, a cytokeratin or epithelial membrane antigen (EMA); (b) a lung tissue marker, such as EpCAM, CA9, CA12, DSG3, FAT2, GPR87, KISS1R, LYPD3, SLC7A11, TMPRSS4, CD133, prominin-1, AC133, programmed death-1 receptor (PD1) or FAS; (c) a breast tissue marker, such as E-cadherin, epithelial membrane antigen (EMA), human epidermal growth factor receptor type 2 (Her2/neu), αvβ6 integrin, EpCAM, carcinoembryonic antigen (CEA), folate receptor-alpha (FR-α), urokinase-type plasminogen activator receptor (uPAR) or placental-specific protein 1 (PLAC1); (d) a liver cell marker, such as CD133, Prominin-1, CD44, EpCAM, delta-like 1 non-canonical Notch ligand 1 (DLK1), ALDH, CD13, CD90, CD24, OV6, ICAM-1, CD34, C-kit, α2δ1, K19, LGR5, GPC3, Annexin A2, CD15, ABC transporters, Nope, DCLK1, ASGPR, CK or CD47; (e) a glioblastoma biomarker, such as fibronectin, CD63, HSP70, Annexin A2, CD9, CD81, CD44, GRP78, CD133, CD15, a sialoglycoprotein, SLC1A3, PTPRZ1, GPR56, CLU or ALD1A3; (f) a urinary tract cell marker, such as a tetraspanin, CD9, CD81, LAMP-1, CD10, CD24, CD44 or CD63; or (g) an amyloid marker such as beta-amyloid precursor protein (β-APP), presenilin protein PS-1, or proinsulin protein PS-2.

In some of the above embodiments, the antibodies are attached to a matrix and the matrix comprises particles such as beads, pellets, or chips, which are optionally magnetic. In some embodiments, the biological fluid sample comprises tears, saliva, lymph fluid, urine, serum, cerebral spinal fluid, pleural effusion, ascites, or plasma. In some embodiments, the sample is serum or plasma.

In some of the above processes, the subject is human. In some embodiments, the subject may be a cancer subject or suspected cancer subject. In some embodiments, the cancer is a solid tumor. In some embodiments, the cancer is breast cancer, triple negative breast cancer, lung cancer, NSCLC, SCLC, liver cancer, urinary tract cancer, bladder cancer, brain cancer, or glioblastoma. In some embodiments, the subject may be an infectious disease subject or suspected infectious disease subject.

In some of the above processes, the cell surface marker comprises EpCAM, EGFR, PSMA, GSP64, CD3, CD49b, CD87, CD95, E-Cadherin CA9, CA12, N-cadherin, OB-cadherin, cadherin-11, cytokeratins and epithelial membrane antigen (EMA). Some processes may further include a second biomarker that comprises one or more of IL-4, IL-5, IL-8, IL-10, IL-35, IFN-α, IFN-y, CXCL10 (IP-10), C reactive protein, hemagglutinin, virus RNA, virus DNA, viral proteins (such as hepatitis B surface antigen, and HIV p24 antigen), bacterial RNA, bacterial DNA, a bacterial protein, a surface immunoglobulin or a receptor for C3 complement component. In some embodiments, the cell surface marker is EpCAM.

In some of the above embodiments, the subject may be an inflammatory disease subject or suspected inflammatory disease subject. In some embodiments, the second biomarker comprises one or more of TNF-α, IL-16, IL-17, IL-21, IL-22, IL-33, CD86, CD80, CRP, IL-1, IL-1α, IL-1β, IL-2, IL-6, IL-8, IL-12, IFN-y, serum amyloid (SAA), COX2, NK-_{K}B, CCL2, CXCL10, CCL2, CXCL5, CXCL9, CXCL6, MMP-7, MMP-2, MMP-9 or CSFIR; and optionally, wherein the cell surface marker is an epithelial cell surface marker, such as EpCAM, EGFR, PSMA, GSP64, CD3, CD49b, CD87, CD95, E-Cadherin CA9, CA12, N-cadherin, OB-cadherin, cadherin-11, a cytokeratin or epithelial membrane antigen (EMA).

In some embodiments, the subject may be an amyloid disease subject or suspected amyloid disease subject. In some such embodiments, the cell surface marker is β-APP, PS-1, or PS-2, and/or wherein the second biomarker is one or more of CD3, CD4, CD8, CD40L, CD45RO, CD45RA, sCD40L, Fas/CD95, CD14, APP, CD19, CD69, IL-2, IFN-α,sCD40, CD11b, CD14, Ibal, CD11b, CD11c, RCA-1, MHCII (including HLA-DR), ferritin, IL-1α, CD68, CD163, Ricinus communis agglutin-1 (RCA-1), translocator protein (TSPO), triggering receptor expressed on myeloid cells 2 (TREM2), CR3 or CD33.

Some of the processes above further comprise isolating total extracellular vesicles (EVs) from the sample, such as by membrane capture or differential ultracentrifugation, determining total protein levels in the isolated total extracellular vesicles, and optionally comparing the total protein levels from the total EVs to levels of cell surface marker in the EVs. In some embodiments, isolation of antibody bound EVs from unbound EVs is through attachment of the antibodies to a matrix. In some embodiments, isolation of the antibody bound EVs from unbound EVs comprises separating the matrix from supernatant in the sample, such as by allowing the matrix to precipitate by gravity or centrifugation.

In some cases, the above processes have one or more of the following characteristics: (a) the sample is not subjected to chromatography either before or after contacting the sample with the antibodies; (b) the sample is not treated before contacting the sample with the antibodies other than optionally to remove particles above 1 µm, above 2 µm, above 5 µm, above 10 µm, or above 20 µm in diameter from the sample and/or to remove cellular bodies and debris larger than EVs; (c) the EVs are not subjected to chromatography either before or after contacting the sample with the antibodies; (d) the antibodies are attached to a matrix, and the matrix is not a filter, an ion-exchange medium, or a membrane; or (e) the antibodies are attached to a matrix and the matrix is not charged.

In some embodiments, the isolation of the cell-type specific EVs from the sample may consist essentially of (a) contacting the sample with antibodies specific for the cell surface marker for the one or more cell types (optionally wherein the antibodies are attached to a matrix), (b) and isolating the antibody-bound EVs from unbound EVs the sample, (c) resuspending the antibody-bound EVs in a lysis buffer, and (d) contacting the EVs with a detection agent specific for a second biomarker.

In some embodiments, the detection agent is an antibody. In some embodiments, the second biomarker may be a protein, a polynucleotide (e.g. an RNA molecule), a lipid, a drug, or a drug metabolite.

In some cases, the subject has cancer or is suspected of having cancer and the processes include a second biomarker that is a polypeptide expressed or overexpressed in tumor cells. In some such embodiments, the processes further include a second biomarker that is a cytokine, cytokine receptor, or an immune checkpoint regulator. In some such embodiments, the second biomarker is one or more of PD-L1, SCF, IL-3, GM-CSF, G-CSF, M-CSF, TNF-alpha, IL-2, IL-5, TMB, CTLA-4, ICOS, 4-1BB (CD137), PD-1, CTLA-4, LAG-3, Tim-3, CD39, IFN-β, IFN-y, IL-2, IL-10, TGF-β, CCR10, CXCR4, CCR7, sMICA, IL-8, IDO1, GBP1, class II MHC molecules, CXCL9, CXCL10 (IP-10), CXCL11, IL-6, CCL4, CCL5, IFNGR1, IFNGR2, JAK2, IRF1, IFIT1, IFIT2, MTAP, miR3, SOCA1, PIAS4, GZMA, GZMB, PRF1, HLA-DQA1, HLA-DRB1, IFNG, STAT1, ICAM1-5, VCAM-1, JAK1, JAK2, CCR5, HLA-DRA, CXCR6, TIGIT, CD27, CD274, PDCD1LG2 (PD-L2), LAG3, NKG7, PSMB10, CMKLR1, CD8A, CD8B, HLA-DB1, HLA-E, CD276, CD3D, CD3E, CD3G, CD247, ZAP70, CD2, CD28, ICOS, IL12Rb1, GZMM, FLTSLG, IL-15, Eotazin, GRO-1, VEGF, HGF, AFP, BCR-ABL, BRCA1/BRCA2, B-Raf V600E, CA-125, CA19-9, CEA, EGFR, HER-2/neu, KIT, PSA, S100, KRAS, UGT1A1 or CD20. In some embodiments, the second biomarker comprises PD-L1. In some embodiments, the tumor cell surface marker is EpCAM and the second biomarker comprises PD-L1.

This disclosure also relates to processes of identifying tumor-derived extracellular vesicles (EVs) in a subject, comprising: (a) providing a plasma sample from the subject, optionally wherein the sample is from 100 µl to 1 mL, 200 µl to 1 mL, from 250 µl to 1 mL, from 100 µl to 500 µl, from 200 µl to 500 µl, from 200 µl to 400 µl, from 250 µl to 500 µl, or from 250 µl to 400 µl; (b) contacting the plasma sample with antibodies specific for EpCAM, optionally wherein the antibodies are attached to a matrix; (c) isolating EpCAM antibody-bound EVs from unbound EVs the sample; (d) optionally resuspending the EpCAM antibody-bound EVs in a lysis buffer; and (e) contacting the EpCAM antibody-bound EVs with an antibody specific for PD-L1 and optionally determining the level of PD-L1 in the EpCAM antibody-bound EVs. This disclosure also relates to processes of determining treatment for a subject with cancer, comprising: (a) providing a plasma sample from the subject, optionally wherein the sample is 100 µl to 1 mL, from 200 µl to 1 mL, from 250 µl to 1 mL, from 100 µl to 500 µl, from 200 µl to 500 µl, from 200 µl to 400 µl, from 250 µl to 500 µl, or from 250 µl to 400 µl; (b) contacting the plasma sample with antibodies specific for EpCAM, optionally wherein the antibodies are attached to a matrix; (c) isolating antibody-bound extracellular vesicles (EVs) from unbound EVs the sample; (d) optionally resuspending the EpCAM antibody-bound EVs in a lysis buffer; (e) contacting the EVs with an antibody specific for PD-L1; (f) determining the level of PD-L1 in the EpCAM antibody-bound EVs; and (g) determining whether the subject should receive treatment with an immune checkpoint inhibitor (e.g. a PD-1 or PD-L1 inhibitor) based on the determined level of PD-L1 in the EpCAM antibody-bound EVs, wherein increases in PD-L1 levels correlate with need for treatment with the inhibitor. In some embodiments, these processes may include determining whether the subject should receive a PD-L1 inhibitor such as atezolimumab, durvalumab, avelumab, envafolimab, BMS-936559, CK-301, CS-1001, SHR-1316, CBT-502, or BGB-A333. In some embodiments, the process may include determining whether the subject should receive a PD-1 inhibitor such as nivolumab, pembrolizumab, cemiplimab, spartalizumab, camrelizumab, sintilimab, tislelizumab, toripalimab, AMP-224, or AMP-514, or a CTLA-4 inhibitor such as ipilimumab. In some cases, the subject is a human having or suspected of having breast cancer, triple negative breast cancer, lung cancer, NSCLC, SCLC, liver cancer, urinary tract cancer, bladder cancer, brain cancer, or glioblastoma. In some embodiments, the process further comprises administering an immune checkpoint inhibitor such as a PD-1 or PD-L1 inhibitor to the subject.

In any of the processes described above, prior to contacting the sample with the antibodies specific to the cell surface marker, the sample can have been treated to remove particles above 1 µm, above 2 µm, above 5 µm, above 10 µm, or above 20 µm in diameter and/or to remove cellular bodies and debris larger than EVs.

In any of the processes described above, the EVs can have a particle size of less than 1 micron, such as 40 nm to less than 1 micron, 40-500 nm, 40-300 nm, 100-400 nm, or 100-300 nm, or have a mean particle size of 150-200 nm.

The present disclosure also provides kits for enriching or identifying cell-type specific extracellular vesicles (EVs) in a biological fluid sample from a subject. In some embodiments, the kits may comprise antibodies specific for a cell surface marker attached to a matrix, and optionally further comprising: (a) one or more detection reagents for detection of a second biomarker, (b) one or more buffers for resuspending antibody-bound EVs and/or for detection of a second biomarker, and (c) instructions for use in enriching EVs from a biological fluid sample of a subject. In some embodiments, the matrix comprises particles such as beads, pellets, or chips, which are optionally magnetic. In some embodiments, the kits may enrich or identify EVs from epithelial cells, lung tissue cells, breast tissue cells, liver tissue cells, prostate tissue cells, kidney tissue cells, urinary tract cells, neural cells, tumor cells, solid tumor cells, lung tumor cells, breast tumor cells, liver tumor cells, prostate tumor cells, kidney tumor cells, urinary tumor cells, glioblastoma cells, or amyloid-beta expressing cells.

In some of the kits described herein, the cell surface marker is: (a) an epithelial cell surface marker, such as EpCAM, EGFR, PSMA, GSP64, CD3, CD49b, CD87, CD95, E-Cadherin CA9, CA12, N-cadherin, OB-cadherin, cadherin-11, a cytokeratin or epithelial membrane antigen (EMA); (b) a lung tissue marker, such as EpCAM, CA9, CA12, DSG3, FAT2, GPR87, KISS1R, LYPD3, SLC7A11, TMPRSS4, CD133, prominin-1, AC133, programmed death-1 receptor (PD1) or FAS; (c) a breast tissue marker, such as E-cadherin, epithelial membrane antigen (EMA), human epidermal growth factor receptor type 2 (Her2/neu), αvβ6 integrin, EpCAM, carcinoembryonic antigen (CEA), folate receptor-alpha (FR-α), urokinase-type plasminogen activator receptor (uPAR) or placental-specific protein 1 (PLAC1); (d) a liver cell marker, such as CD133, Prominin-1, CD44, EpCAM, delta-like 1 non-canonical Notch ligand 1 (DLK1), ALDH, CD13, CD90, CD24, OV6, ICAM-1, CD34, C-kit, α2δ1, K19, LGR5, GPC3, Annexin A2, CD15, ABC transporters, Nope, DCLK1, ASGPR, CK or CD47; (e) a glioblastoma biomarker, such as fibronectin, CD63, HSP70, Annexin A2, CD9, CD81, CD44, GRP78, CD133, CD15, a sialoglycoprotein, SLC1A3, PTPRZ1, GPR56, CLU or ALD1A3; (g) a urinary tract cell marker, such as a tetraspanin, CD9, CD81, LAMP-1, CD10, CD24, CD44 or CD63; or (h) an amyloid marker such as beta-amyloid precursor protein (β-APP), presenilin protein PS-1, or proinsulin protein PS-2.

In some kits herein, the second biomarker comprises a protein, a polynucleotide (e.g. an RNA molecule), a lipid, a drug, or a drug metabolite. In some kits, the second biomarker is one or more of PD-L1, SCF, IL-3, GM-CSF, G-CSF, M-CSF, TNF-alpha, IL-2, IL-5, TMB, CTLA-4, ICOS, 4-1BB (CD137), PD-1, CTLA-4, LAG-3, Tim-3, CD39, IFN-β, IFN-y, IL-2, IL-10, TGF-β, CCR10, CXCR4, CCR7, sMICA, IL-8, IDO1, GBP1, class II MHC molecules, CXCL9, CXCL10 (IP-10), CXCL11, IL-6, CCL4, CCL5, IFNGR1, IFNGR2, JAK2, IRF1, IFIT1, IFIT2, MTAP, miR3, SOCA1, PIAS4, GZMA, GZMB, PRF1, HLA-DQA1, HLA-DRB1, IFNG, STAT1, ICAM1-5, VCAM-1, JAK1, JAK2, CCR5, HLA-DRA, CXCR6, TIGIT, CD27, CD274, PDCD1LG2 (PD-L2), LAG3, NKG7, PSMB10, CMKLR1, CD8A, CD8B, HLA-DB1, HLA-E, CD276, CD3D, CD3E, CD3G, CD247, ZAP70, CD2, CD28, ICOS, IL12Rb1, GZMM, FLTSLG, IL-15, Eotazin, GRO-1, VEGF, HGF, AFP, BCR-ABL, BRCA1/BRCA2, B-Raf V600E, CA-125, CA19-9, CEA, EGFR, HER-2/neu, KIT, PSA, S100, KRAS, UGT1A1, CD20, TNF-α, IL-16, IL-17, IL-21, IL-22, IL-33, CD86, CD80, CRP, IL-1, IL-1α, IL-1β, IL-2, IL-6, IL-8, IL-12, IFN-y, serum amyloid (SAA), COX2, NK-xB, CCL2, CXCL10, CCL2, CXCL5, CXCL9, CXCL6, MMP-7, MMP-2, MMP-9, CSFIR, CD3, CD4, CD8, CD40L, CD45RO, CD45RA, sCD40L, Fas/CD95, CD14, APP, CD19, CD69, IL-2, IFN-α, sCD40, CD11b, CD14, Ibal, CD11b, CD11c, RCA-1, MHCII (including HLA-DR), ferritin, IL-1α, CD68, CD163, Ricinus communis agglutin-1 (RCA-1), translocator protein (TSPO), triggering receptor expressed on myeloid cells 2 (TREM2), CR3 or CD33.

### BRIEF DESCRIPTION OF THE FIGURES

The figures of the present application were previously provided in color in the priority US provisional application, which is incorporated herein by reference.

FIGS. 1A-1B show differential expression of EpCAM in various cancers. FIG. 1A shows RNA-seq data from 7366 human tissues was collected by The Cancer Genome Atlas (TCGA) and was processed and visualized using GeneHub, a Genentech tool. EpCAM expression is displayed in normalized reads per kilobase of exon model per million mapped reads (nRPKM). Crosshatched bars indicate there is significantly higher expression of EpCAM (by log₂fold change) between tumor and normal samples. EpCAM levels in tumors are shown to the right of levels in normal tissue in the lanes for breast, cervix, colon, head/neck, kidney, liver, lung, pancreas, and prostate. EpCAM levels in tumors are also shown for adrenal, leukemia, lymphoid, and ovary. FIG. 1B shows RNA-seq dataset from FIG. 1A was used to focus on EpCAM expression in commonly used subtypes of lung cancer and breast cancer. EpCAM expression is much higher in lung adenocarcinomas than squamous lung cancers, and is uniformly high across breast cancer subtyped by PAM50.

FIG. 1C shows a Flowchart for EpCAM+ EV enrichment. A Plasma/PBS mixture was loaded onto the CellSearch® platform, which positively selects for EpCAM+ EVs via magnetic bead attachment. The EVs were then lysed with RIPA buffer and total protein levels were tested via BCA assay. PD-L1 levels were then measured using the Quanterix® Simoa® PD-L1 assay. Total EV harvesting: Plasma was spun at 100,000 g for 3 hours, after which the supernatant was removed and the pellet washed with PBS and spun at 100,000 g for 1 hour. The resultant pellet was then lysed with RIPA buffer and total protein levels tested via BCA assay. PD-L1 levels were then measured using the Quanterix® Simoa® assay.

FIG. 2 shows EpCAM expression from a set of solid tumors and normal tissue (microarray) (expression in tumors is shown to the left of expression in normal tissue for each cancer type in the graph). Microarray data from Gene Logic consists of 862 unpaired tumor and normal tissues. EpCAM gene expression is shown as log₂(Probe Intensity). * indicates p value is significant and *** indicates p value is highly significant (p < 0.001) using a one-tailed T-Test with unequal variance.

FIGS. 3A-3D show transmission electron microscopy analysis of NSCLC, TNBC and healthy donor plasma demonstrates EVs of various sizes in respective fractions. FIG. 3A shows a brief flowchart for isolation of Total EVs by DUC used for TEM analysis. The 20 K and 100 K fractions were sequentially collected and stored in PBS before being imaged. For FIG. 3B, NSCLC plasma from two individuals was pooled, and the 20 K and 100 K fractions were imaged, along with the EV-depleted supernatant. The majority of the 100 K fraction was used for CellSearch®, to generate EpCAM+ EVs, which were also imaged with TEM (under “Output”). For FIGS. 3C-3D, the process in 3A repeated with pools of triple-negative breast cancer plasma (FIG. 3C) and healthy donor plasma from two individuals (FIG. 3D).

FIGS. 4A-4F show that Nanosight® tracking analysis confirms the presence of vesicles within the expected size range of EVs in cancer patient plasma. The input for EM (total EVs isolated from non-small cell lung cancer (NSCLC) (FIGS. 4A and 4D), triple negative breast cancer (TNBC) (FIGS. 4B and 4E), and healthy donor plasma, EV depleted plasma (FIGS. 4C and 4F) was run with Nanosight® tracking analysis. EV size data for 20 K fractions are shown in FIGS. 4A-C and EV size data for 100 K fractions are shown in FIGS. 4D-F. In all 20 K fractions, the size range is 50 nm - 1 um (many vesicles around 250 nm). In 100 K fractions, the size range is 50-250 nm, with most particles below 200 nm in size.

FIGS. 5A-D show custom Quanterix® PD-L1 assays that demonstrate that EpCAM+ EVs are specific to cancer patient plasma. FIG. 5A shows a schematic for two PD-L1 assays. PD-L1 sample is incubated with atezolizumab prior to analysis. After assay initiation on the Quanterix®, magnetic beads conjugated with anti-PD-L1 Ab are used for capture. An antibody against the atezolizumab framework conjugated with biotin serves as the detection. Finally, streptavidin beta galactosidase is used for the detection on the HD-1. FIG. 5B shows a flow chart of the assays. Specifically, 12 mL of plasma was split into two aliquots of 6 mL each, which were used for Total EV and EpCAM+ EV isolation. Total protein in these subsets was quantitated using BCA assay. EpCAM EVs were isolated using CellSearch®, while Total EVs were isolated using DUC. Levels of PD-L1 protein from EpCAM+ EVs were measured using the custom drug tolerant PD-L1 assay. FIG. 5C shows protein concentration in Total EVs and EpCAM+ EVs and percent protein in EpCAM+ EVs vs. Total EVs in various tissues. FIG. 5D shows PD-L1 concentration in EpCAM+ EVs from various tissues.

FIGS. 6A-6E show PD-L1 standard curves and that PD-L1 is detectable in Total EVs from cancer and healthy donors. FIGS. 6A and 6B show the standard curve for two custom PD-L1 assays, high sensitivity PD-L1 (FIG. 6A) and drug tolerant PD-L1 assay (FIG. 6B). The y-axis shows average enzyme per bead (AEB), and a 4-parameter logistic curve was used to fit both curves. FIGS. 6C-6D show absolute (FIG. 6C) and normalized (FIG. 6D) PD-L1 levels for a subset of cancerous and healthy donors (normalization to total protein). These samples show good linearity in PD-L1 concentration assayed across a wide range of dilution factors. FIG. 6E shows the linearity of PD-L1 concentration in total EVs from several tissues.

FIGS. 7A-7F show that the isolation of EpCAM+ EVs from cancer cell lines H1975, BT474, and A549 is reproducible and specific to EpCAM. FIG. 7A shows EpCAM and PD-L1 expression, as measured using RNA-seq (nRPKM values). FIGS. 7B-7C show total EVs extracted (using DUC) from 50 mL of supernatant from these cell lines. The resulting pellet of EVs was resuspended in PBS with 5% glycerol and brought to 6 mL with HDP. EpCAM+ EVs were isolated using the CellSearch®, and imaged with TEM. FIG. 7D shows the PD-L1 to total protein ratio in lysates of the EV pellets from FIG. 7B. The EV pellets were resuspended in 200 uL of RIPA buffer and lysed and the lysate was tested using the Quanterix® PD-L1 assay. FIG. 7E shows total protein concentration and PD-L1 concentration in total EVs. The EVs were isolated using DUC on H1975 supernatant and spiked into 3 separate aliquots of HDP, and run on the CellSearch® separately. Total protein was measured using BCA assay, and the Quanterix® PD-L1 assay was used to assess accuracy. For FIG. 7F, equal parts H1975 EVs and Jurkat EVs were added to HDP and run on the CellSearch®. The resulting EVs showed reduced PD-L1 concentration.

FIGS. 8A-D show the precision of Quanterix® PD-L1 assay and NTA of total EVs from cancer cell lines. FIG. 8A shows PD-L1 concentration in three aliquots of healthy plasma spiked with EVs taken from H1975 cells and split into three aliquots to test precision. Each identical aliquot was put through the CS/Simoa® PD-L1 process, showing that variance across aliquots was low. FIGS. 8B-8D show total EVs (100 K fraction) in different cancer cell lines H1975 (FIG. 8B), A549 (FIG. 8C), and MDA-MB-231 (FIG. 8D). EVs were analyzed with Nanosight® to determine particle size.

FIGS. 9A-9D show PD-L1 assay performance with different % of RIPA and various starting volumes. In FIG. 9A, concentrated H1975 EVs were resuspended in 10% or 80% RIPA, and lysed. This lysate was run using the Quanterix® PD-L1 assay, and 4-paramenter standard curves are overlaid. FIG. 9B shows indicated amounts of H1975 EVs that were brought up to 3 mL of HDP and run on the CellSearch®. BCC = back-calculated, and % difference between the dilutions is shown. In FIG. 9C, concentrated BT474 EVs were used to ascertain a reliable limit of detection, which was 1.85 pg/mL. PD-L1 5D1 antibody was added in one case, to block the complex formation and show low detection. Additional NSCLC EV lysate read above the limit of detection. FIG. 9D shows that PD-L1 levels are linear and specific in various mixtures of EVs from H1975 and BT474 cell lines.

FIGS. 10A-10B show additional linearity and precision of the PD-L1 assay. In FIG. 10A, varying amounts of healthy plasma spiked with EVs extracted from a EpCAM high, PD-L1 high NSCLC cell line (H1975) were used on the CellSearch® platform to test linearity. Linearity showed an R² value of 0.996 when assayed for PD-L1. In FIG. 10B, healthy plasma was spiked with EVs taken from H1975 cells and run on CellSearch®. The aliquot was tested using the Quanterix® PD-L1 assays on Day 1 and then on Day 2 and the percentage difference determined.

### DETAILED DESCRIPTION

### I. Definitions

Unless otherwise defined, scientific and technical terms used in connection with the present invention shall have the meanings that are commonly understood by those of ordinary skill in the art.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As used herein, the transition term “consisting essentially of,” when referring to steps of a claimed process signifies that the process comprises no additional steps beyond those specified that would materially affect the basic and novel characteristics of the process. As used herein, the transition term “consisting essentially of,” when referring to a composition or product, such as a kit, signifies that it comprises no additional components beyond those specified that would materially affect its basic and novel characteristics.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As utilized in accordance with the present disclosure, the following terms, unless otherwise indicated, shall be understood to have the following meanings:

A “biological fluid sample” or “sample” as used herein refers to any biological fluid from a subject in which may contain EVs, such as tears, saliva, lymph fluid, urine, serum, cerebral spinal fluid, pleural effusion, ascites, and plasma. A sample may be taken directly from a subject, or may be pre-treated in some embodiments to remove large debris.

“Extracellular vesicles (EVs)” herein comprise sub-cellular particles that may be shed or secreted from cells, including tissue cells and tumor cells, into biological fluids. EVs comprise exosomes, microvesicles, and apoptotic bodies, for example.

EVs that are “cell-type specific” are primarily derived from particular types of cells or are EVs that are enriched for those derived from particular types of cells. The cell types in some embodiments can be based on function (e.g. epithelial cells or fibroblasts), location (e.g. from particular tissues), or based on disease state (e.g. tumor cells, inflamed cells, or the like). EVs can be enriched for cell types such as, for example, epithelial cells or cells that express epithelial cell surface markers, or such as, for example, tumor cells like solid tumor cells or cells that express tumor-associated cell surface markers, or, for example, cells from particular tissues such as breast or lung and the like, or tumor cells from organs such as breast, lung, prostate, etc. EVs that are cell-type specific may be obtained by using a particular cell surface marker as a means of selection.

The term “marker” or “biomarker” as used herein refers to an indicator, e.g., predictive, diagnostic, and/or prognostic, which can be detected in a sample. The marker or biomarker may be a protein or polypeptide or nucleic acid molecule as well as a lipid or glycolipid or a drug or drug metabolite. The biomarker may serve as an indicator of a particular subtype of a disease or disorder (e.g., cancer, Alzheimer’s disease, or an inflammatory disease) characterized by certain, molecular, pathological, histological, and/or clinical features. A biomarker may be at least partly exposed on the surface of EVs or present in the lumen of EVs. A “cell surface marker,” for example, is a type of biomarker that is at least partially exposed on the surface of cells, such as a membrane-spanning protein or a glycolipid, and is thus also expected to be at least partially exposed on the surface of EVs from cells in which it is present. A “tumor biomarker” or “tumor marker,” as used herein, refers to a protein, polypeptide, nucleic acid, lipid, glycolipid, drug, drug metabolite, or other molecule that is enriched in tumor cells and that may, in turn, be enriched in EVs derived from tumor cells compared to EVs from other cells.

A “cell surface marker,” for example, may be used to mark EVs that may originate from particular cell types, such as cells of one or more tissues, as opposed, for example, to EVs originating from blood or hematologic cells or non-diseased cells. Thus, in some embodiments, a “cell surface marker” comprises a protein or other molecule that is enriched on the surface of cells from one or more particular types, such as cells having a particular function (e.g. fibroblast or epithelial cells), or cells from one or more particular bodily tissues or organs, or diseased cells, compared to EVs originating from other cells, and that, in some embodiments, may serve to identify EVs coming from certain specific types of cells such as cells having a particular function, coming from a particular tissue type or organ, or diseased cells. In some embodiments, a “cell surface marker” is an “epithelial cell surface marker,” which, as used herein, refers to a marker that is enriched on the surface of fibroblast or epithelial cells and tumors of fibroblast cell or epithelial cell origin. In some aspects, when more than one biomarker is used in an assay, the second or additional biomarker (or biomarkers) may be referred to as a “second biomarker” or “secondary biomarker” to distinguish it from the “cell surface marker.”

A “matrix” as used herein refers to a substrate to which an antibody may be bound. Examples of matrices that could be used in the processes herein include various types of particles of any shape or form, including beads, pellets, or chips, as well as sheets, resins, or surfaces, and the like, which could be made from various materials capable of binding to proteins such as antibodies. In some embodiments, the matrix may comprise magnetic particles, such as magnetic beads, pellets, or chips, for example, which may facilitate separation of the matrix from a solution.

The terms “isolating EVs” or “enriching EVs” or similar terms, as used in describing the processes herein, mean that the EVs that are isolated or enriched (e.g. EVs bound to an antibody) are physically separated in some fashion from unbound EVs in the sample. For example, EVs may be isolated or enriched if they possess a cell surface marker to which an antibody used in the processes herein is specific. Binding to the antibody may separate the bound EVs from the unbound EVs, thus enriching the bound EVs, for example, so that they can be further assayed.

A “detection agent” is used herein in the broadest sense to mean an agent or molecule that is employed to detect the presence of a biomarker associated with EVs, such as a biomarker located in the lumen of or on the surface of EVs. In some embodiments, the “detection agent” may comprise a “binding agent,” which is a molecule that is capable of specifically binding to a particular biomarker. Examples of binding agents include antibodies, nucleic acid probes (including RNA, DNA, peptide nucleic acid, and other nucleic acid molecules capable of hybridizing to a target sequence), aptamers, and other molecules that may specifically bind to a biomarker molecule. In some embodiments, detection agents may be attached to one or more other molecules, such as a secondary antibody, nucleic acid probes, biotin, a second matrix, and/or a labeling molecule, for example, to allow for detection of the marker and/or for amplification of the signal from the marker.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. Antibodies used for detection of biomarkers herein, for example, may include a variety of modifications in order to allow binding of antibody to biomarker to be detected. For example, an antibody may be attached to a matrix, such as a matrix particle, an antibody may be attached to one or more other detection agents such as a secondary antibody, nucleic acid probes, biotin, a second matrix, and/or a labeling molecule.

An “effective amount” of an agent, e.g., a pharmaceutical composition, refers to an amount effective, at dosages and for periods of time necessary, to achieve the desired therapeutic or prophylactic result.

The terms “polypeptide” and “protein” are used interchangeably and refer to a polymer of amino acid residues. Such polymers of amino acid residues may contain natural and/or non-natural amino acid residues, and include, but are not limited to, peptides, oligopeptides, dimers, trimers, and multimers of amino acid residues. The terms also include polymers of amino acids that have modifications such as, for example, glycosylation, sialylation, and the like, or that are complexed with other molecules. Protein biomarkers herein include, for example, native and heterologous proteins such as proteins enriched in disease or mutated in disease cells, such as oncogenic proteins, bacterial proteins, viral proteins, and the like, as well as protein drugs and protein drug metabolites.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Nucleic acid biomarkers herein include, for example, deoxyribonucleic acid (DNA) including e.g., genomic DNA, mitochondrial DNA, methylated DNA, and the like, and ribonucleic acid (RNA), in particular messenger RNA (mRNA), and other cellular RNA molecules such as small interfering RNA (siRNA), micro RNA (miRNA), non-coding RNAs, as well as heterologous nucleic acids such as viral DNA or RNA or bacterial DNA or RNA, or drugs and metabolites that comprise DNA or RNA.

In general, a “subject” as referred to herein is an individual whose biological fluid sample is to be tested for presence of EVs. In some embodiments, the subject is a human. However, in some embodiments, the subject may also be another mammal, such as a domestic or livestock species, e.g., dog, cat, rabbit, horse, pig, cow, goat, sheep, etc., or a laboratory animal, such as a mouse or rat. Mammals include, but are not limited to, domesticated animals (e.g., cows, sheep, cats, dogs, and horses), primates (e.g., humans and non-human primates such as monkeys), rabbits, and rodents (e.g., mice and rats), for example.

A subject herein may have “cancer” or may be suspected to have “cancer.” Cancers herein may include, for example, solid tumors, which comprise tumors originating from tissue cells of the body. In some embodiments, a subject may have or be suspected to have a cancer such as breast cancer (including triple negative breast cancer), lung cancer (including small cell lung cancer or non-small cell lung cancer, adenocarcinoma of the lung, and squamous carcinoma of the lung), prostate cancer, testicular cancer, penile cancer, esophageal cancer, tumors of the biliary tract, brain cancer (including glioblastoma), colorectal cancer, colon cancer, rectal cancer, kidney cancer (including renal cell carcinoma), liver cancer (hepatoma), adrenal cancer, cervical cancer, uterine cancer, endometrial cancer, vulval cancer, salivary gland carcinoma, squamous cell cancer of the head and neck, leukemia, lymphoma, lymphoid cancer, ovarian cancer, pancreatic cancer, bladder cancer, skin cancer such as melanoma, and urinary tract cancer. In some embodiments, the cancer is breast cancer. In some embodiments, the cancer is lung cancer, such as small cell lung cancer (SCLC) or non-small cell lung cancer (NSCLC). In some embodiments, the cancer is urinary tract cancer, such as bladder cancer. In some embodiments, the cancer is brain cancer such as glioblastoma. In some embodiments, the cancer is liver cancer (hepatoma).

As used herein, “treatment” (and grammatical variations thereof such as “treat” or “treating”) refers to clinical intervention in an attempt to alter the natural course of a disease in the individual being treated, and can be performed, for example, during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, clinical interventions are used to delay development of a disease or to slow the progression of a disease.

### Ii. General Components and Methods

This disclosure provides, inter alia, processes for enriching extracellular vesicles (EVs) from biological fluid samples from mammalian subjects.

Extracellular vesicles (EVs) can comprise particles such as exosomes, which are about 40-100 nm, microvesicles, which are about 100 nm to 1 micron, and apoptotic bodies, which can be about 1-5 microns. Exosomes arise from intraluminal vesicles, which are byproducts of the endocytic process and exist in the cytoplasm. Once these precursors are secreted from the plasma membrane of cells, they become exosomes. Microvesicles (sometimes called ectosomes) are assembled directly at the plasma membrane and may contain similar cargo and surface markers as exosomes (such as tetraspanins). In general, EVs can comprise heterogeneous mixtures of these sub-particles from various cell types. In some aspects, the EVs herein have a mean particle size of 150-200 nm. In some aspects, the EVs have a particle size of less than 1 micron, such as 40 nm to less than 1 micron, 40-500 nm, 40-300 nm, 100-400 nm, or 100-300 nm.

The samples can include any bodily fluid that may comprise EVs. Examples include tears, saliva, lymph, urine, serum, cerebral spinal fluid, pleural effusion, ascites, and plasma. The choice of sample may depend on the cellular source of the EVs the researcher wishes to study. For example, EVs from bladder or kidney cells may appear in urine, while EVs from various epithelial tissues may appear in serum or plasma. In some embodiments, the biological fluid sample does not comprise whole blood.

In some embodiments, sample have volumes of from 100 µl to 7 mL, from 200 µl to 6.5 mL, from 250 µl to 6.5 mL, from 200 µl to 6 mL, from 250 µl to 6 mL, from 200 µl to 5 mL, from 200 µl to 4 mL, from 200 µl to 3 mL, from 200 µl to 2 mL, from 100 µl to 1 mL, from 150 µl to 1 mL, from 200 µl to 1 mL, from 250 µl to 1 mL, from 200 µl to 500 µl, from 200 µl to 500 µl, from 200 µl to 400 µl, from 250 µl to 500 µl, or from 250 µl to 400 µl. In some embodiments, samples have volumes of 100 µl, 150 µl, 200 µl, 250 µl, 300 µl, 350 µl, 400 µl, 500 µl, 600 µl, 700 µl, 800 µl, 900 µl, or 1 mL, or a range bounded by any two of those volumes. In contrast, sample volumes for detecting substances such as circulating tumor cells in whole blood, for example, are significantly larger, such as 7.5 mL or at least 6.5 mL. For example, automated devices for detecting circulating tumor cells in whole blood may require at least 6.5 mL or at least 7.5 mL samples. Thus, where serum or plasma is used as a sample, significantly smaller volumes may be needed for the processes herein of analyzing markers on EVs in comparison to processes of analyzing markers on circulating tumor cells, which may result in smaller blood draws from subjects.

In some embodiments herein, a sample can be pre-treated before incubation with the antibodies to remove larger cell debris, including larger apoptotic bodies (e.g. of about 2-5 microns), for example, via a pre-spin or centrifugation process step. In some embodiments, a sample is pre-treated to remove particles above a certain size, such as above 1 µm (1 micron), above 2 µm, above 5 µm, above 10 µm, or above 20 µm. For example, the sample can be run through a filter that holds back particles above a particular size. In other embodiments, the sample is not pre-treated to remove such larger particles or debris but is used directly in the process. In some embodiments, a sample can be pre-treated by mixing it with another substance such as a preservative or buffer. While in other embodiments, the sample is not pre-treated in such a way. In general, the above sample volumes refer to the sample volumes prior to any such pre-treatment steps, if such steps affect the sample volume. In some embodiments, the sample could be pre-treated using a chromatography step, e.g., cation or anion exchange or size-exclusion or gel filtration or affinity chromatography or could be subjected to such a step after EVs are isolated. In other embodiments, the sample is not pre-treated using any chromatography step. In some embodiments, the sample is not subjected to chromatography either before or after contacting the sample with the antibodies. In some embodiments, the sample is not subjected to chromatography either before contacting the sample with the antibodies or after isolating the EVs.

In some embodiments, the sample is contacted with antibodies specific for a cell surface marker, optionally wherein the antibodies are attached to a matrix. A matrix herein to which the antibodies are attached may be of a variety of types, such as particles of any shape, such as beads, pellets, or chips, so long as the particles can be attached to the antibodies. In some embodiments, a matrix may be a surface, such as a chip or plate to which the antibodies are attached. In some embodiments, the antibodies may be attached to a particular location or section of a surface, for example. In some embodiments, the matrix may have properties that readily allow it to be separated from the sample so that material from the sample that is recognized by the antibodies remains bound to the matrix via the antibodies and is separated from the rest of the sample. In this way, separating the antibodies specific for the cell surface marker from the rest of the sample will isolate the antibody-bound EVs from the rest of the sample, thus enriching those EVs. EVs that have been isolated by antibody binding may then, in some embodiments, be further studied, for example, by assaying for the presence of one or more second biomarkers, which might be specific to particular types of cells, including abnormal cells such as tumor cells or the like, or which might be specific to particular biological processes such as inflammation or drug metabolism.

In some embodiments herein, the sample is merely contacted with or mixed with the antibodies attached to the matrix, and then the matrix is separated from the sample. Thus, in some embodiments, no wash steps are employed. In some embodiments, for example, the antibody-attached matrix and the sample are mixed in solution. In some embodiments, therefore, the matrix is not in the form of a column through which the sample must flow. For example, in some embodiments, the matrix is not an affinity column, but is instead a set of particles, such as a powder composed of particles, or an emulsion of particles, such as a slurry or resin, or is a surface that is put into contact with the sample and then removed. In some embodiments, the matrix can be removed by particular physical properties. For example, magnetic matrices allow for ready separation from samples on the basis of their magnetic properties. Thus, in some embodiments, the matrix may comprise magnetic particles or surfaces to allow ease of separation from the sample after the matrix comprising the antibodies has been contacted with the sample. In some embodiments, the contacting of the sample with the antibody-matrix, and the subsequent separation of the matrix from the sample, resulting in isolation of the antibody-bound EVs on the matrix, can be performed in an instrument that takes advantage of physical properties of the matrix such as magnetism. For example, in some embodiments, these steps of the process may be performed in a CellSearch® system (Menarini Silicon Biosystems, Italy).

In some embodiments, the antibodies used to detect the cell surface marker are attached to a matrix covalently. In other embodiments, they may be attached noncovalently. In some embodiments, the antibodies may be indirectly attached to the matrix through other intervening or linking molecules. For example, antibodies may be first attached to a molecule such as biotin or streptavidin, which may, in turn, be recognized by a binding partner on the surface of a matrix particle.

In some embodiments, the matrix is made of a substance that is generally inert toward EVs so that EVs bound to the antibody-matrix will primarily be those bound to the antibodies on the matrix rather than to the matrix itself. In some embodiments, the matrix is not a filter. In some embodiments, the matrix is not a membrane such as a cellulose or polymer-based membrane. In some embodiments, the matrix is not a chromatography medium, such as an ion exchange medium. In some embodiments, the matrix is not a filter, membrane, or ion exchange medium. In some embodiments, the matrix is not charged but instead has neutral pH.

In some embodiments where the antibodies specific for cell surface markers are attached to a matrix, isolation of antibody-bound EVs from unbound EVs can be performed using the matrix. For example, in some embodiments the isolation can occur by separating the matrix from the rest of the sample (i.e., the supernatant), such as by allowing the matrix comprising EV-bound antibodies to precipitate by gravity and removing the supernatant, or by using centrifugation such as in a spin-column to speed the process of precipitation. In other embodiments, antibody-bound EVs can be isolated from unbound EVs, for example, by adding reagents to which the antibodies bind, such as secondary antibodies attached to a matrix, for example, and then using the added reagents as a means for separation of bound EVs from unbound EVs in the sample.

In some embodiments it may be desirable to determine how many EVs collected in particular biological fluid samples come from a particular type of cell, such as an epithelial cell type, or a particular tissue type (e.g. liver, kidney, breast) compared to EVs from other types of cells such as blood or immune cells. One method of obtaining the percentage of EVs coming from a particular cell or tissue type is to compare the approximate number of EVs isolated in the present methods to the number of EVs isolated by another technique that is not cell-type or tissue specific, such as differential ultracentrifugation (DUC) or size exclusion chromatography (SEC), or membrane capture. For example, EVs isolated by the present methods as well as EVs isolated by DUC or SEC can be tested for their relative total protein contents. Comparison of the total protein contents from EVs isolated using the cell surface markers and antibodies herein vs. isolated using DUC or SEC may indicate the percentage of EVs in the sample that stem from cells expressing the chosen cell surface markers.

### A) Cell Surface Markers

A variety of cell surface markers may be used, as discussed further below. Cell surface markers, in some embodiments, are chosen as proteins or glycolipids or other molecules that are at least partially exposed on cellular membranes, and thus, that should also be at least partially exposed on the membranes of EVs. In some embodiments, where EVs from a particular cell type are to be detected, a molecule that is enriched on the surface of that cell type may be chosen. This may help to distinguish EVs from that cell type from other EVs. In some embodiments, once antibody-bound EVs are isolated from unbound material in the sample, such as using a matrix containing the antibody-bound EVs, additional biomarker tests may be run to identify EVs from particular cell types or to detect the presence of abnormal cell types.

In some embodiments, the cell surface marker is a lung tissue or lung cancer biomarker. Examples of a lung tissue or lung cancer biomarker include, without limitation, EpCAM, CA9, CA12, DSG3, FAT2, GPR87, KISS1R, LYPD3, SLC7A11, TMPRSS4, CD133, prominin-1, AC133, programmed death-1 receptor (PD1), and FAS.

In some embodiments, the cell surface marker is a breast tissue or breast cancer biomarker. Examples of a breast tissue or breast cancer biomarker include, without limitation, E-cadherin, epithelial membrane antigen (EMA), human epidermal growth factor receptor type 2 (Her2/neu), αvβ6 integrin, EpCAM, carcinoembryonic antigen (CEA), folate receptor-alpha (FR-α), urokinase-type plasminogen activator receptor (uPAR) and placental-specific protein 1 (PLAC1).

In some embodiments, the cell surface marker is a liver cell or liver cancer biomarker. Examples of a liver cell or liver cancer biomarker include, without limitation, CD133, Prominin-1, CD44, EpCAM, delta-like 1 non-canonical Notch ligand 1 (DLK1), ALDH, CD13, CD90, CD24, OV6, ICAM-1, CD34, C-kit, α2δ1, K19, LGR5, GPC3, Annexin A2, CD15, ABC transporters, Nope, DCLK1, ASGPR, CK and CD47.

In some embodiments, the cell surface marker is a glioblastoma biomarker. Examples of a glioblastoma biomarker include, without limitation, fibronectin, CD63, HSP70, Annexin A2, CD9, CD81, CD44, GRP78, CD133, CD15, sialoglycoproteins, SLC1A3, PTPRZ1, GPR56, CLU and ALD1A3.

In some embodiments, the cell surface marker is a urinary tract cancer biomarker. Examples of a urinary tract biomarker include, without limitation, tetraspanins, CD9, CD81, LAMP-1, CD10, CD24, CD44 and CD63.

In some embodiments, the cell surface marker is an epithelial marker. Examples of epithelial markers include, without limitation, EpCAM, EGFR, PSMA, GSP64, CD3, CD49b, CD87, CD95, E-Cadherin CA9, CA12, N-cadherin, OB-cadherin, cadherin-11, cytokeratins and epithelial membrane antigen (EMA).

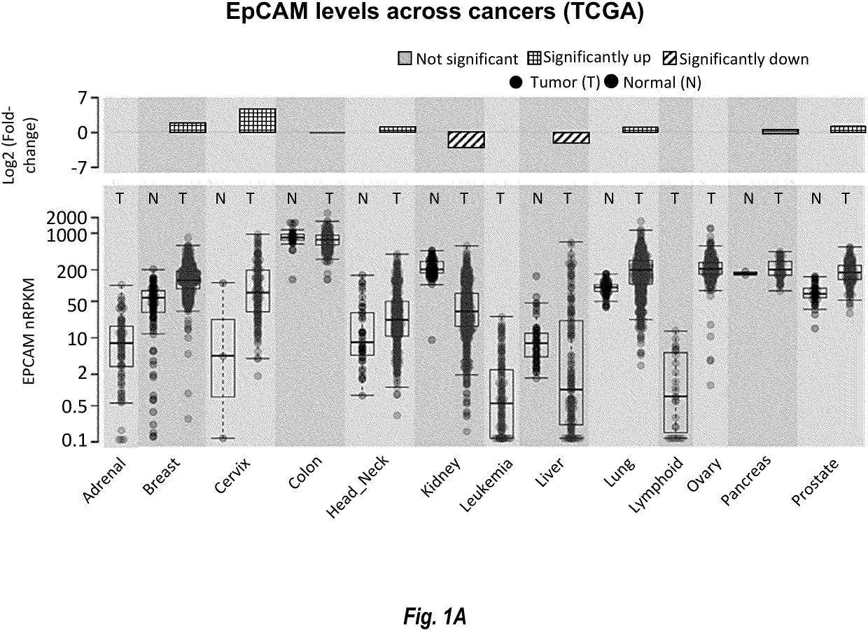

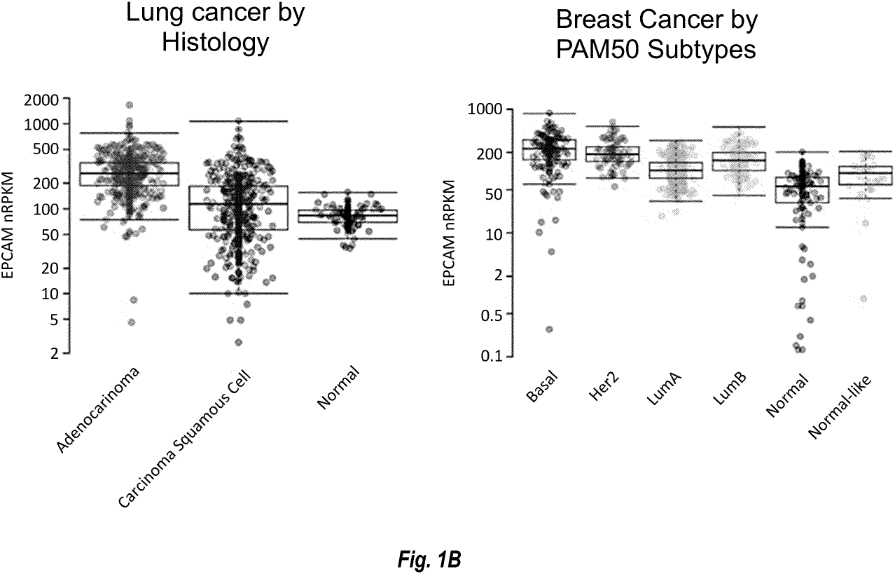

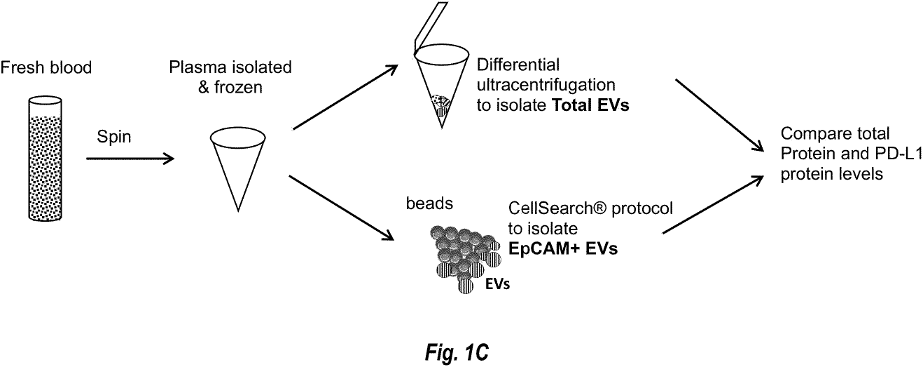

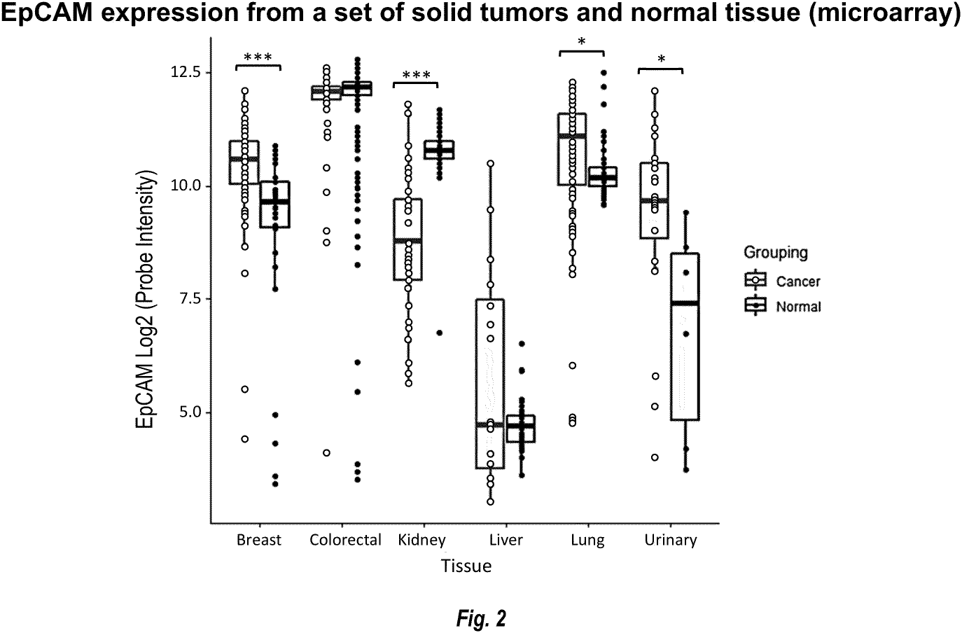

In some embodiments, the cell surface marker is a protein that is enriched in diseased cells of a particular tissue, such as prostate specific antigen (PSA) to detect prostate cell-derived EVs, particularly those from prostate tumor cells; thyroid transcription factor 1 (TTF-1) for lung tissue cells; or EpCAM, the expression of which may be enriched in EVs from tumor cells in breast, lung, urinary tract, head and neck, prostate and liver cancers, for example, in comparison to normal breast, lung, urinary tract, head and neck, prostate and liver tissues. (See FIGS. 1A and 2.)

### B) Secondary Biomarkers

In some embodiments, after EVs have been isolated, the EVs are characterized further by assaying for the presence of or level of at least one second or secondary biomarker. Such additional biomarkers may be located either on the surface of EVs, or they may be located in the lumen of EVs.

Prior to detecting a second biomarker, the EVs isolated using antibodies specific for the cell surface marker may be first resuspended in a solution allowing for detection of the second biomarker. Where the secondary biomarker or biomarkers are located in the EV lumen, for instance, EVs that have been isolated using the antibody specific for the cell surface marker (i.e. cell surface marker antibody-bound EVs) may be lysed prior to the step of detecting the additional biomarker or biomarkers. For example, in some embodiments, the EVs may be resuspended in a lysis buffer for this purpose.

In some embodiments, one or more washes of the isolated EVs may be performed prior to detecting a second biomarker. In other embodiments, no such washes are necessary and presence of a second biomarker is detected by simply adding a solution comprising appropriate detection agents to the cell surface marker antibody-bound EVs.

Choice of a secondary marker for analysis depends on the purpose of isolating the EVs. For example, if the purpose is to detect tumor cells or evaluate tumor cells in a subject, the secondary biomarker may be a tumor-associated biomarker such as a cytokine or chemokine, growth factor receptor, immune checkpoint regulator or the like. Examples include PD-L1, SCF, IL-3, GM-CSF, G-CSF, M-CSF, TNF-alpha, IL-2, IL-5, TMB, CTLA-4, ICOS, 4-1BB (CD137), PD-1, CTLA-4, LAG-3, Tim-3, CD39, IFN-β, IFN-y, IL-2, IL-10, TGF-β, CCR10, CXCR4, CCR7, sMICA, IL-8, IDO1, GBP1, class II MHC molecules, CXCL9, CXCL10 (IP-10), CXCL11, IL-6, CCL4, CCL5, IFNGR1, IFNGR2, JAK2, IRF1, IFIT1, IFIT2, MTAP, miR3, SOCA1, PIAS4, GZMA, GZMB, PRF1, HLA-DQA1, HLA-DRB1, IFNG, STAT1, ICAM1-5, VCAM-1, JAK1, JAK2, CCR5, HLA-DRA, CXCR6, TIGIT, CD27, CD274, PDCD1LG2 (PD-L2), LAG3, NKG7, PSMB10, CMKLR1, CD8A, CD8B, HLA-DB1, HLA-E, CD276, CD3D, CD3E, CD3G, CD247, ZAP70, CD2, CD28, ICOS, IL12Rb1, GZMM, FLTSLG, IL-15, Eotazin, GRO-1, VEGF, HGF, AFP, BCR-ABL, BRCA1/BRCA2, B-Raf V600E, CA-125, CA19-9, CEA, EGFR, HER-2/neu, KIT, PSA, S100, KRAS, UGT1A1 and CD20. If the purpose is to detect for the presence of inflammation, a secondary biomarker may include markers of inflammation such as TNF-α, IL-16, IL-17, IL-21, IL-22, IL-33, CD86, CD80, CRP, IL-1, IL-1α, IL-1β, IL-2, IL-6, IL-8, IL-12, IFN-y, serum amyloid (SAA), COX2, NK-_{K}B, CCL2, CXCL10, CCL2, CXCL5, CXCL9, CXCL6, MMP-7, MMP-2, MMP-9 and CSFIR.

In some cases, a secondary biomarker may be a protein that is expressed in or on cells of a particular tissue or associated with a particular disease. In some cases, the biomarker is expressed in the cytosol of a cell and thus, can be expected to be found within the lumen of EVs. In some cases, a secondary biomarker may be a nucleic acid molecule that is expressed in such cells, such as an mRNA molecule or siRNA molecule. In some cases, a secondary molecule is a heterologous molecule, such as a molecule that is expressed by an infectious agent such as a virus, bacteria, or the like. In some cases, a secondary biomarker may be a drug or a byproduct of a drug such as a drug metabolite, to check for presence of the drug or drug activity in cells.

In any of the above embodiments, there may be one or more secondary biomarkers. For example, further characterizations may be performed by assaying a third, or fourth, even further biomarker.

### III. Exemplary Uses of the Methods

Because the processes herein are compatible with a variety of biological fluid samples, cell surface markers, and secondary biomarkers, they may be used in a wide variety of settings. Some examples are provided in this section. These are merely examples and are not meant to be limiting in any way.

### A) Tumor-Associated EVs

In some embodiments, the methods herein may be used to detect EVs associated with tumor cells. Thus, in some embodiments, the subject either has cancer or is suspected of having cancer, or the process is used as part of a screening procedure to help diagnose whether the subject has cancer or has a recurrence of cancer. In some embodiments, depending on the markers used, the process can also be used to help determine whether a subject with cancer should receive a particular type of drug regimen, for example, by assaying for the presence of particular cell surface markers or second biomarkers in the EVs that correlate with responsiveness to a particular drug regimen.

In some embodiments, the subject has, is suspected of having, or is being screened for presence of a solid tumor. In some embodiments, the subject has, is suspected of having, or is being screened for presence of a cancer selected from breast cancer (including triple negative breast cancer), lung cancer (including small cell lung cancer or non-small cell lung cancer, adenocarcinoma of the lung, and squamous carcinoma of the lung), prostate cancer, testicular cancer, penile cancer, esophageal cancer, tumors of the biliary tract, brain cancer (including glioblastoma), colorectal cancer, colon cancer, rectal cancer, kidney cancer (including renal cell carcinoma), liver cancer (hepatoma), adrenal cancer, cervical cancer, uterine cancer, endometrial cancer, vulval cancer, salivary gland carcinoma, squamous cell cancer of the head and neck, leukemia, lymphoma, lymphoid cancer, ovarian cancer, pancreatic cancer, bladder cancer, skin cancer such as melanoma, and urinary tract cancer.

In some embodiments, the cell surface marker is a protein that is enriched in tumor cells of a particular tissue in comparison to normal tissues, such as prostate specific antigen (PSA) to detect prostate cell-derived EVs, particularly those from prostate tumor cells, or thyroid transcription factor 1 (TTF-1) for lung tissue cells, or EpCAM, the expression of which may be enriched in EVs from tumor cells in, for instance, breast, lung, urinary tract, head and neck, prostate and liver cancers, for example, in comparison to normal breast, lung, urinary tract, head and neck, prostate and liver tissues. (see FIGS. 1A and 2.)

In some embodiments, the cancer is breast cancer, such as triple negative breast cancer. Cell surface markers that may be used to isolate and enrich EVs from breast tissues, and thus breast tumor cells include EpCAM, EGFR, E-cadherin, epithelial membrane antigen (EMA), human epidermal growth factor receptor type 2 (Her2/neu), αvβ6 integrin, EpCAM, carcinoembryonic antigen (CEA), folate receptor-alpha (FR-α), urokinase-type plasminogen activator receptor (uPAR) and placental-specific protein 1 (PLAC1).

In some embodiments, the cancer is lung cancer, such as small cell lung cancer (SCLC) or non-small cell lung cancer (NSCLC). Cell surface markers that may be used to isolate and enrich EVs from lung tissues, and thus from lung tumor cells, include EpCAM, CA9, CA12, CXorf61, DSG3, FAT2, GPR87, KISS1R, LYPD3, SLC7A11, TMPRSS4, CD133, prominin-1, AC133, and FAS.

In some embodiments, the cancer is liver cancer or hepatoma, and the cell surface marker is a liver cell or liver cancer biomarker, such as CD133, Prominin-1, CD44, EpCAM, delta-like 1 non-canonical Notch ligand 1 (DLK1), ALDH, CD13, CD90, CD24, OV6, ICAM-1, CD34, C-kit, α2δ1, K19, LGR5, GPC3, Annexin A2, CD15, ABC transporters, Nope, DCLK1, ASGPR, CK and CD47.

In some embodiments, the cancer is glioblastoma and the cell surface marker is a glioblastoma biomarker, such as fibronectin, CD63, HSP70, Annexin A2, CD9, CD81, CD44, GRP78, CD133, CD15, sialoglycoproteins, SLC1A3, PTPRZ1, GPR56, CLU and ALD1A3.

In some embodiments, the cancer is a urinary tract cancer such as bladder cancer, or urinary cancer, and the cell surface marker is a urinary tract cancer biomarker such as tetraspanins, CD9, CD81, LAMP-1, CD10, CD24, CD44 and CD63.

Once EVs are enriched from tissue, the tissue may then be assessed for the amount of a cytokine or other tumor biomarker, such as PD-1, PD-L1, SCF, IL-3, GM-CSF, G-CSF, M-CSF, TNF-alpha, IL-2, IL-5, TMB, CTLA-4, ICOS, 4-1BB (CD137), PD-1, CTLA-4, LAG-3, Tim-3, CD39, IFN-β, IFN-y, IL-2, IL-10, TGF-β, CCR10, CXCR4, CCR7, sMICA, IL-8, IDO1, GBP1, class II MHC molecules, CXCL9, CXCL10 (IP-10), CXCL11, IL-6, CCL4, CCL5, IFNGR1, IFNGR2, JAK2, IRF1, IFIT1, IFIT2, MTAP, miR3, SOCA1, PIAS4, GZMA, GZMB, PRF1, HLA-DQA1, HLA-DRB1, IFNG, STAT1, ICAM1-5, VCAM-1, JAK1, JAK2, CCR5, HLA-DRA, CXCR6, TIGIT, CD27, CD274, PDCD1LG2 (PD-L2), LAG3, NKG7, PSMB10, CMKLR1, CD8A, CD8B, HLA-DB1, HLA-E, CD276, CD3D, CD3E, CD3G, CD247, ZAP70, CD2, CD28, ICOS, IL12Rb1, GZMM, FLTSLG, IL-15, Eotazin, GRO-1, VEGF, HGF, AFP, BCR-ABL, BRCA1/BRCA2, B-Raf V600E, CA-125, CA19-9, CEA, EGFR, HER-2/neu, KIT, PSA, S100, KRAS, UGT1A1 and CD20.

In some embodiments, the secondary biomarker is a lung cancer biomarker, such as SCF, IL-5, CRP and fibrinogen.

In some embodiments, the secondary biomarker is a breast cancer biomarker, such as IL-3, GM-CSF, CRP and SAA.

In some embodiments, the secondary biomarker is a liver cancer biomarker, such as IL-1, IL-6 and CRP.

In some embodiments, the secondary biomarker is a glioblastoma biomarker, such as IL-6, TNF-α and CRP.

In some embodiments, the secondary biomarker is a urinary tract cancer biomarker such as CRP, GM-CSF, M-CSF, CXCR4 and IL-6.

In some embodiments, level of PD-L1 is measured as a secondary biomarker, as part of a screening procedure for cancer, for instance, to help determine if treatment with an immune checkpoint inhibitor should be provided, or to help assess the presence or state of the cancer. In some cases, the cancer is lung cancer (including SCLC and NSCLC), melanoma, Hodgkin lymphoma, bladder cancer, kidney cancer, breast cancer (including triple negative), cervical cancer (including cervical squamous cell carcinoma and endocervical adenocarcinoma), gastric/gastroesophagealjunction (GEJ) adenocarcinoma, head and neck squamous cell carcinoma, urothelial carcinoma, ovarian cancer, colorectal cancer and esophageal squamous cell carcinoma (ESCC).

The PD-L1 level in EVs may then be used, for example, to detect the presence of tumor-derived EVs, as the PD-L1 level is expected to be higher in cancer subjects than in healthy subjects, and also to determine if a subject may be in need of therapy with an immune checkpoint inhibitor. Thus, in some embodiments, the process may be used to help determine if a cancer subject needs treatment with a PD-L1 inhibitor such as atezolimumab, durvalumab, avelumab, envafolimab, BMS-936559, CK-301, CS-1001, SHR-1316, CBT-502, or BGB-A333, or with a PD-1 inhibitor such as nivolumab, pembrolizumab, cemiplimab, spartalizumab, camrelizumab, sintilimab, tislelizumab, toripalimab, AMP-224, or AMP-514, or a CTLA-4 inhibitor such as ipilimumab. In some embodiments, the process may be used during or after treatment with an immune checkpoint inhibitor such as a PD-1/PD-L1 inhibitor or CTLA-4 inhibitor. For example, the PD-L1 level could be re-checked during and after a course of treatment, such as, to determine if the dosage or frequency of the drug should be adjusted or whether the drug needs to be re-administered.

In some embodiments, the level of a second biomarker such as a cytokine, or immune checkpoint regulator such as PD-L1, SCF, IL-3, GM-CSF, G-CSF, M-CSF, TNF-alpha, IL-2, IL-5, TMB, CTLA-4, ICOS, 4-1BB (CD137), PD-1, CTLA-4, LAG-3, Tim-3, CD39, IFN-β, IFN-y, IL-2, IL-10, TGF-β, CCR10, CXCR4, CCR7, sMICA, IL-8, IDO1, GBP1, class II MHC molecules, CXCL9, CXCL10 (IP-10), CXCL11, IL-6, CCL4, CCL5, IFNGR1, IFNGR2, JAK2, IRF1, IFIT1, IFIT2, MTAP, miR3, SOCA1, PIAS4, GZMA, GZMB, PRF1, HLA-DQA1, HLA-DRB1, IFNG, STAT1, ICAM1-5, VCAM-1, JAK1, JAK2, CCR5, HLA-DRA, CXCR6, TIGIT, CD27, CD274, PDCD1LG2 (PD-L2), LAG3, NKG7, PSMB10, CMKLR1, CD8A, CD8B, HLA-DB1, HLA-E, CD276, CD3D, CD3E, CD3G, CD247, ZAP70, CD2, CD28, ICOS, IL12Rb1, GZMM, FLTSLG, IL-15, Eotazin, GRO-1, VEGF, HGF, AFP, BCR-ABL, BRCA1/BRCA2, B-Raf V600E, CA-125, CA19-9, CEA, EGFR, HER-2/neu, KIT, PSA, S100, KRAS, UGT1A1 or CD20, is compared against levels of the biomarker observed in prior cancer subjects with known disease severity, such as tumor grade or stage or presence of metastasis. Such prior subjects may be used to create a sort of standard curve or range that allows one to determine the correlation of expression of a particular biomarker in EVs and, for example, parameters such as tumor stage or grade, risk of metastasis, tumor growth, and risk of recurrence following treatment. Such correlations allow mapping of a subject’s biomarker expression level in EVs against that of other subjects, which allow predictions to be made as to the degree of severity of the particular subject’s cancer stage, grade, or growth rate, and the risk of recurrence or metastasis following treatment, for example.

In some embodiments, the methods herein may be used in assessing treatment options, and the methods may further comprise administering a particular treatment to a subject on the basis of the level of second biomarker found in the subject’s EVs. Thus, for example, the level of PD-L1 in the subject’s EVs can be used as basis for starting treatment with an immune checkpoint inhibitor such as a PD-1/PD-L1 inhibitor or CTLA-4 inhibitor, or for increasing dose or frequency of such a treatment or for decreasing dose or frequency of such a treatment. These methods facilitate patient selection for targeted therapy.

### B) Infectious Disease, Inflammatory Disease, and Amyloid Disease-Associated EVs

In some embodiments, the methods herein may be used to detect EVs associated with other diseases or diseased cells. For example, cell surface markers associated with particular tissues or types of tissue (e.g. epithelial tissue) can be used to isolate EVs from those tissues. And additional biomarkers that may be found in or on EVs associated with, for example, inflammatory diseases, infectious diseases, amyloid diseases and other conditions such as Alzheimer’s disease, and the like, can then be assayed.

Thus, in some aspects, the subject has an inflammatory disease or is suspected of having an inflammatory disease. Examples of inflammatory diseases include as rheumatoid arthritis (including methotrexate-resistant rheumatoid arthritis), systemic lupus erythematosus (lupus; including methotrexate-resistant lupus), asthma (including methotrexate-resistant asthma), multiple sclerosis, juvenile chronic arthritis, spondyloarthropathies, systemic sclerosis (scleroderma), idiopathic inflammatory myopathies (dermatomyositis, polymyositis), Sjogren’s syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave’s disease. Hashimoto’s thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Bane syndrome, and chronic inflammatory demyelinating polyneuropathy, hepatobiliary diseases such as infectious hepatitis (hepatitis A, B, C, D. E and other non-hepatotropic viruses), autoimmune chronic active hepatitis, primary biliary cirrhosis, granulomatous hepatitis, and sclerosing cholangitis, inflammatory bowel disease (IBD). including ulcerative colitis: methotrexate-resistant IBD, Crohn’s disease, methotrexate-resistant Crohn’s disease gluten-sensitive enteropathy, and Whipple’s disease, autoimmune or immune-mediated skin, diseases including bullous skin diseases, erythema multiforme and contact dermatitis, psoriasis (including methotrexate-resistant psoriasis), allergic diseases include as asthma, allergic rhinitis, atopic dermatitis, food hypersensitivity and urticaria, immunologic diseases of the lung such as eosinophilic pneumonia, idiopathic pulmonary fibrosis and hypersensitivity pneumonitis, transplantation associated diseases including graft rejection and graft-versus-host-disease; fibrosis, including kidney fibrosis and hepatic fibrosis, cardiovascular disease, including atherosclerosis and coronary artery disease, cardiovascular events associated with chronic kidney disease, myocardial infarction, and congestive heart failure, diabetes, including type II diabetes, Bronchiolitis obliterans with organizing pneumonia (BOOP). hemophagocytic syndrome, macrophage activation syndrome, sarcoidosis, and periodontitis. In some embodiments, the cell surface marker is an epithelial marker, such as EpCAM, EGFR, PSMA, GSP64, CD3, CD49b, CD87, CD95, E-Cadherin CA9, CA12, N-cadherin, OB-cadherin, cadherin-11, cytokeratins and epithelial membrane antigen (EMA). In some embodiments, the secondary marker is an inflammatory marker, such as TNF-α, IL-16, IL-17, IL-21, IL-22, IL-33, CD86, CD80, CRP, IL-1, IL-1α, IL-1β, IL-2, IL-6, IL-8, IL-12, IFN-γ, serum amyloid (SAA), COX2, NK-κB, CCL2, CXCL10, CCL2, CXCL5, CXCL9, CXCL6, MMP-7, MMP-2, MMP-9 and CSFIR, which may be expressed differently in normal tissues versus in the presence of inflammation.

In some embodiments, the subject has an infectious disease or is suspected of having an infectious disease. Examples of infectious diseases include viral diseases (such as AIDS (HIV infection), hepatitis A, B, C. D, and E, herpes), bacterial infections, fungal infections, protozoal infections and parasitic infections. In these cases, the cell surface marker is an epithelial marker, such as EpCAM, EGFR, PSMA, GSP64, CD3, CD49b, CD87, CD95, E-Cadherin CA9, CA12, N-cadherin, OB-cadherin, cadherin-11, cytokeratins and epithelial membrane antigen (EMA). And the secondary marker may be a molecule that is particularly associated with presence of the disease, such as a viral, bacterial, fungal, or protozoal protein, or a toxin produced by the infectious agent. In some embodiments, the secondary marker is an infectious disease marker, such as IL-4, IL-5, IL-8, IL-10, IL-35, IFN-α,IFN-y, CXCL10 (IP-10), C reactive protein, hemagglutinin, virus RNA or DNA, viral proteins (such as hepatitis B surface antigen, and HIV p24 antigen) bacterial RNA or DNA, bacterial proteins, surface immunoglobulins and receptor for C3 complement component.

In some embodiments, the processes herein may be used to characterize EVs from neural tissue or brain tissue, for example, in cerebral spinal fluid. In some embodiments, the cell surface marker and additional biomarkers may be used to help detect the presence of diseased neural cells, for example, cells impacted by amyloid disease. Thus, in some embodiments, the subject has an amyloid disease or is suspected of having an amyloid disease. Examples of amyloid diseases include Alzheimer’s disease, Parkinson’s disease, transmissible spongiform encephalopathy (including Creutzfeldt-Jacob disease), taopathies (including Pick’s disease), Huntington disease, familial British dementia, familial Danish dementia, light chain amyloidosis, heavy chain amyloidosis, AA amyloidosis, familial amyloid polyneuropathy, familial amyloid cardiomyopathy, dialysis related amyloidosis, ApoAI amyloidosis, ApoAII amyloidosis, ApoAIV amyloidosis, ApoCII amyloidosis, Apo CIII amyloidosis, Finish type familial amyloidosis, fibrinogen amyloidosis and diabetes mellitus type 2.

For example, Alzheimer’s disease is thought to be related to the accumulation of mutations in particular genes, the beta-amyloid precursor protein (β-APP), and the presenilin proteins PS-1 and PS-2. All three of these proteins are cell surface proteins (i.e. integral membrane proteins), and thus, are expressed on the surface of cells, meaning that they may also be found on EVs stemming from the cells on which they are expressed. Using β-APP, PS-1, or PS-2 as a cell surface marker, may allow isolation of EVs stemming from neural tissues, allowing the EVs to be further examined for the levels or presence of other, secondary biomarkers in subsequent assays.

In some embodiments, a secondary marker may be used to further characterize the amyloid disease. In some cases, the secondary marker is an Alzheimer’s disease marker, such as CD3, CD4, CD8, CD40L, CD45RO, CD45RA, sCD40L, Fas/CD95, CD14, APP, CD19, CD69, IL-2, IFN-α,sCD40, CD11b, CD14, Ibal, CD11b, CD11c, RCA-1, MHCII (including HLA-DR), ferritin, IL-1α, CD68, CD163, Ricinus communis agglutin-1 (RCA-1), translocator protein (TSPO), triggering receptor expressed on myeloid cells 2 (TREM2), CR3 and CD33.

### C) Uses for Examining Drugs or Drug Metabolites in EVs

In some embodiments, the methods herein may be used to check for the presence of a drug or drug metabolite in EVs, for example, to determine if cells are taking up a drug intended to work within the cytoplasm. Thus, in such embodiments, once a cell surface marker is used to isolate EVs, the drug or a metabolic product of the drug may be assayed as a second biomarker using the processes herein. In other cases, if a drug or metabolite is expected to be found on the cell surface, it could be used as a cell surface marker herein. In other embodiments, the methods herein can be used to check for the presence of a drug by assessing other biomarkers that relate to the drug’s activity. For example, if a drug is expected to increase the concentration of a particular molecule in the cytoplasm or on the cell surface, then that molecule can be used as a cell surface marker and/or second biomarker in methods herein.

Any of the methods herein may be conducted in computer-controlled equipment so that they can be at least partially automated. For example, certain types of matrices, such as particular particle types, may be manipulated in automated or semi-automated systems, such that contact between the matrix particles and the sample may be automatically controlled by a computer and appropriate software. For example, magnetic beads coated with antibodies for a cell surface marker may be manipulated in partially automated systems on the basis of their magnetic properties so that they can be mixed with the sample and then separated from the sample by control of a computer and appropriate software. The methods herein, for example, may be performed in a partially automated fashion with commercial machines or equipment, such as in a CellSearch® system, by modifying the normal system protocol designed for detecting circulating tumor cells. In addition, tests for determining the presence or level of additional, i.e., second, biomarkers in EVs bound to antibody for the cell surface marker may be at least partially automated by allowing a computer and software to determine, for example, the process of allowing the EVs to contact detection reagents for the additional biomarkers and optionally, also to quantitate the level of the biomarkers that is observed.

### IV. Kits

Embodiments herein also include, for example, kits comprising reagents associated with processes herein. In some embodiments, kits are intended for sale to users of the processes that may include some or all of the necessary reagents for characterizing EVs for specific purposes. Kits herein may comprise, for example, antibodies for cell surface markers, a matrix or matrix particles coated with antibodies for cell surface markers. Thus, such antibodies may or may not be pre-attached to a matrix. Kits may comprise, for example, detection reagents for detecting one or more secondary biomarkers in EVs, such as antibodies, nucleic acid molecules, and optionally also color labelling reagents associated with such detection reagents. Kits may also comprise control samples and reagents to be used with control samples. Kits, in some embodiments, may also contain one or more buffers or solutions for washing or resuspension of EV-containing solutions during the process, for example, a resuspension buffer or lysis buffer to resuspend and/or lyse EVs after they have been isolated using the antibodies specific for the cell surface marker so that levels of a second biomarker in the EVs can be determined. In some embodiments, kits may comprise directions for use.

### EXAMPLES

### Example 1

This Example describes a semi-automated method to enrich tumor EVs from human plasma and compare these to Total EVs isolated using differential ultracentrifugation (DUC). The latter consist of all EVs within a certain plasma sample, either originating from cells in circulation or shed from various tissues in the body. To develop a method for tumor EV enrichment, Epithelial Cell Adhesion Molecule (EpCAM) was used as a cell-surface marker. EpCAM is enriched in cancers of epithelial origin (Packeisen et al. 1999; Gastl et al. 2000) and is the target of several clinical-stage molecules (reviewed in Eyvazi et al. 2018). To assess enrichment of tumor EVs, we also developed custom, ultra-sensitive immunoassays that could detect levels of the immune checkpoint protein, programmed death ligand 1 (PD-L1) from EVs within cancer patient plasma and cell culture supernatant.

PD-L1 acts as a “brake” to the immune system, restricting the ability of CD8 T cells to kill tumor cells. PD-L1 can be present on immune cells such as macrophages and T cells, as well as on tumor cells (Tamura et al. 2001; Yamazaki et al. 2002). Tumor expression of PD-L1 by immunohistochemistry is a diagnostic biomarker for anti-PD-L1 therapies such as atezolizumab (Tecentriq™, F. Hoffman-La Roche Ltd.). Atezolizumab is an engineered immunoglobulin monoclonal anti-PD-L1 antibody that blocks binding between PD-L1 and its receptor PD-1, restoring the anti-tumor activity of T cells and enhancing T-cell priming (reviewed in Sun et al., 2018). PD-L1 can be membrane-bound (mPD-L1) and expressed on the surface of cells, or in soluble form (sPD-L1) shed from cells and detected in the peripheral blood of cancer patients (Fest et al. 2013; Takahashi et al. 2016). Data suggest that the extracellular fraction of mPD-L1 is cleaved by matrix metalloproteinases (MMPs) like MMP-7 and MMP-13, releasing sPD-L1 (Dezutter-Dambuyant et al. 2015; Hira-Miyazawa et al. 2018). However, immunoassays detecting sPD-L1 in serum or plasma may also be detecting mPD-L1 on EVs, in cases where the antibodies used in these assays recognize the extracellular domain of PD-L1.

Levels of sPD-L1 protein have been examined in the serum and plasma of cancer patients using enzyme-linked immunosorbent assays (ELISA) (Frigola et al., 2011; Rossille et al., 2014). These reports indicate that sPD-L1 may be prognostic in renal cell carcinoma and diffuse large B-cell lymphoma, respectively.

For this study, we decided to focus on patients with specific subtypes of lung and breast cancers, as these tumors often express high levels of EpCAM, (Willms et al. 2016). Lung cancer is one of the deadliest cancers, with a 5-year survival rate of 18.6% (Noone et al., 2015). Non-small cell lung cancer (NSCLC) represents 85-90% of all lung cancers diagnosed, as opposed to the less frequent small cell lung cancer. Tecentriq™ is currently an approved therapy for metastatic NSCLC patients without EGFR or ALK mutations (Rittmeyer et al. 2017; Socinski et al. 2018). Recently, the combination of Tecentriq™ with chemotherapy led to significant clinical benefit for patients with metastatic or locally advanced triple-negative breast cancer (TNBC) (Schmid et al. 2018). While only 15-20% of all breast cancers are TNBC, this subtype is highly aggressive and characterized by a lack of treatment options (Bianchini et al. 2016). As with many cancers, lung and breast tumor biopsies are hard to acquire, so liquid biopsies are a desired approach.

Here we describe the development of a sensitive EpCAM-based method to enrich for tumor EVs from low volume of human plasma using the CellSearch® platform. We showcase the potential to enrich for subtypes of EVs and to interrogate protein markers from these and other EV subpopulations.

### Materials and Methods

Gene expression analysis: RNA-seq data from 7366 human tissues was collected by The Cancer Genome Atlas (TCGA) and was processed and visualized using GeneHub®, a Genentech tool. Gene expression is displayed in normalized reads per kilobase of exon model per million mapped reads (nRPKM). For tumor types where data from both cancer and normal tissue is available, log₂(Fold change) is shown. Microarray data from Gene Logic consists of 862 tumor and normal tissues. EpCAM gene expression is shown as log₂(Probe Intensity).

Human samples: Fresh whole blood from cancer patients was obtained from BioIVT (Westbury, New York), which operates under IRB-approved protocols. Additionally, blood samples from healthy volunteers were procured from the Genentech Samples for Science program, which was approved by the Western Institutional Review Board. Blood samples were collected in Vacutainer K₂EDTA tubes and inverted at least 10 times to ensure anticoagulant was mixed well. Samples were kept at room temperature and processed within 48 hours of collection. Plasma was obtained by centrifuging whole blood at 400 g for 10 minutes. Plasma was then transferred to labeled polypropylene screw-cap cryovials and frozen at -80° C. until further processing.

Cell lines: A549, BT474, H1975, PC9, and Jurkat clone E6-1 cell lines were maintained and authenticated by the Genentech Cell Bank (gCELL) as described (Yu et al. 2015). For isolating EVs, the cell culture media was removed and pelleted at 300 g for 5 minutes. After this, the supernatant was immediately used in one of the below protocols to isolate either Total or EpCAM+ EVs.

Isolation of Total EVs by Differential Ultracentrifugation (DUC): Plasma or cell culture supernatant was first centrifuged at 300 g for 10 minutes to clear debris. That supernatant was then spun at 20,000 g using a Beckman Coulter Optima XPN-90 (rotor: 50.2 Ti) for 30 minutes. The pellet mostly contains microvesicles and maybe some apoptotic bodies (the majority of which was separated with 2000 g, the speed used to initially separate plasma from whole blood). The supernatant was centrifuged again at 100,000 g for 3 hours to pellet the exosome fraction. For transmission electron microscopy (TEM), the pellets were resuspended in PBS. EVs were lysed in RIPA buffer (Thermo Fisher Scientific) on ice for 30 minutes for protein analysis. Total protein levels were assayed using BCA or CBQCA kit (Thermo Fisher Scientific).

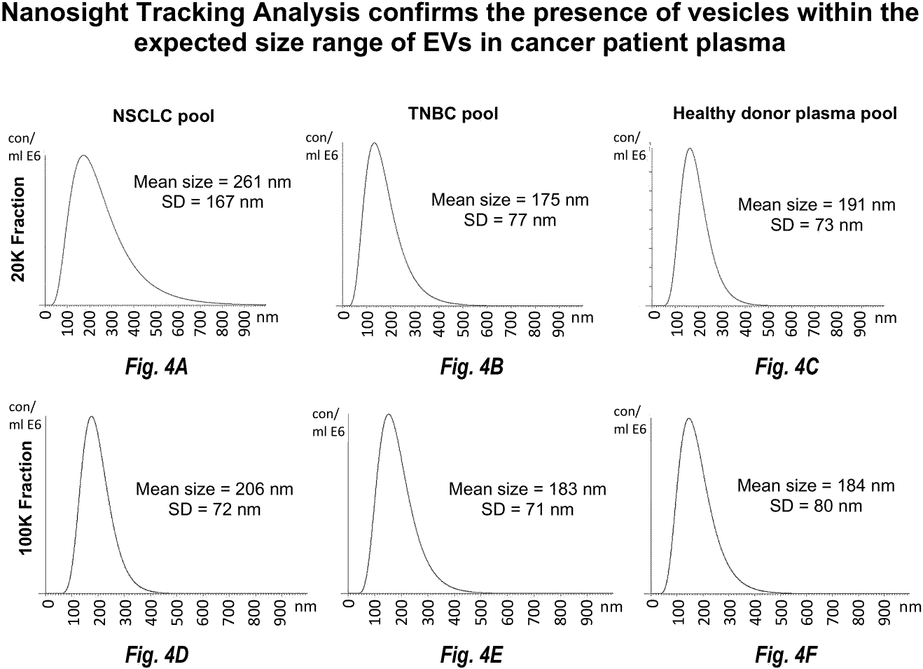

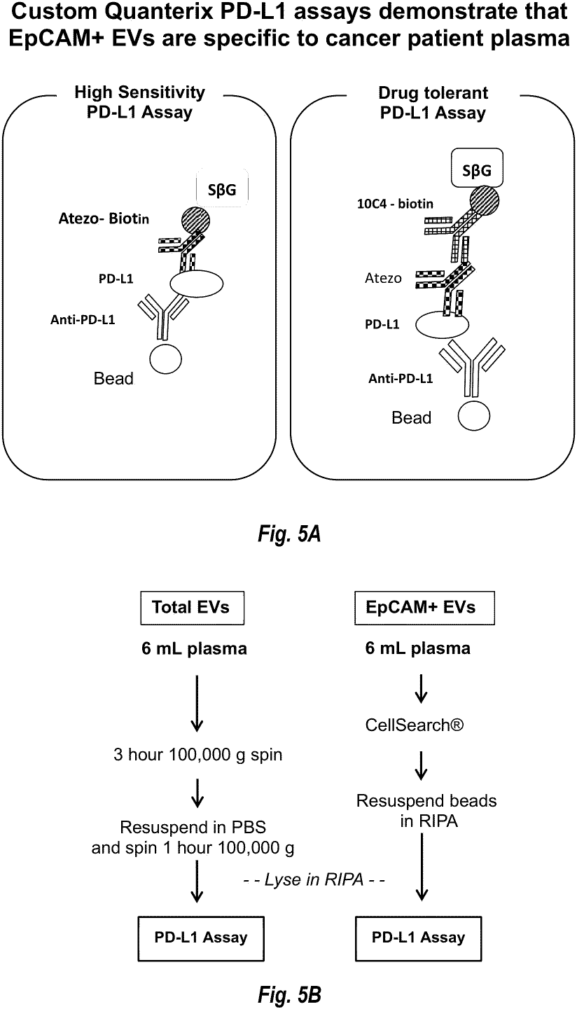

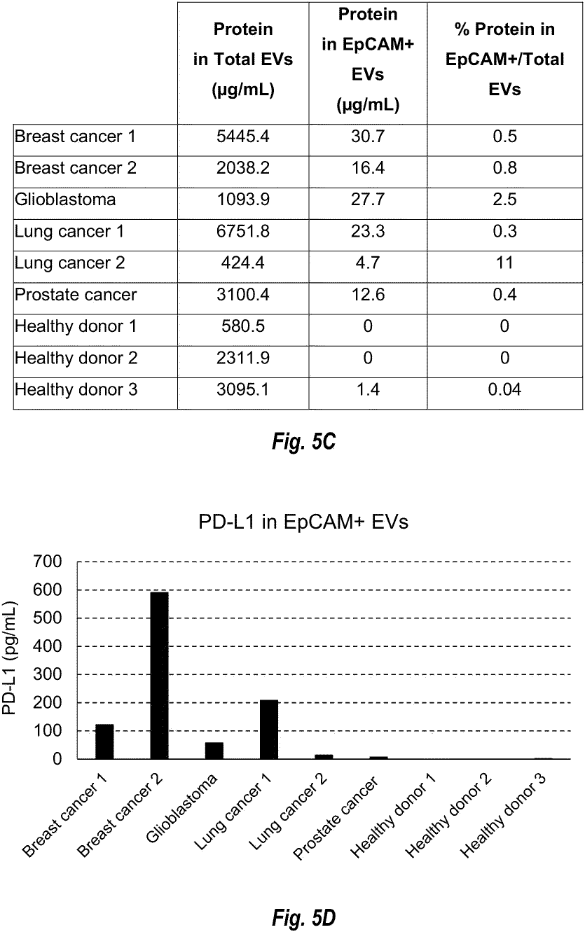

Isolation of EPCAM+ EVs: Plasma was first centrifuged at 300 g for 10 min to clear debris. For data in FIGS. 2 and 3, 6 mL of this pre-spun plasma was run on the CellTracks™ Autoprep machine using a Cellsearch® Profile kit (Menarini Silicon Biosystems, Inc., San Diego, CA). The beads with bound EpCAM+ EVs were lysed in 300 µL of RIPA buffer for 30 minutes on ice. Then, the sample was put on a quadrupole magnet for 10 minutes to separate the supernatant from the beads. The supernatant was used for protein analysis. For data in FIGS. 4 and 5, different volumes of pre-spun plasma were resuspended in PBS up to 6 mL, and run on the CellTracks Autoprep machine.

From cancer cell lines, 50 mL of supernatant was centrifuged with the full DUC protocol to generate a pellet of EVs. The EVs were resuspended in 100 µL of PBS with 5% glycerol, then either frozen at -60° C. or spiked into plasma and processed through the CellSearch® platform. The sample was put on a quadrupole magnet for 10 minutes, and the supernatant was used for protein analysis.

Transmission Electron Microscopy (TEM): EpCAM+ EV samples were isolated using the CellSearch® Profile kit as described above, except that PBS was used to resuspend the EVs using the quadrupole magnets. These samples were adsorbed onto the surface of formvar-and carbon-coated TEM grids for 30 minutes. Grids were quickly rinsed using several drops of distilled water. Negative staining was performed for 1 minute with 1% uranylacetate. After air drying, the grids were analyzed in a JEOL JEM-1400 TEM and imaged with a Gatan ultrascan 1000 digital camera at magnifications from 5000x (overview) to 50000x (individual exosomes).

Nanosight analysis: The Nanosight® LM10 instrument (Malver Panalytical; Amesbury, UK) is used to characterize microparticles based on their size. The instrument was calibrated using silica microspheres of relevant diameters prior to sample analysis. EVs isolated from differential ultracentrifugation (following 20 K spin and 100 K spin) were then reconstituted and diluted 1:100 in PBS. One milliliter of the diluted EVs were injected into the LM10 sample chamber using a syringe pump to ensure even flow. The instrument was equipped with a blue (488 nm) laser. The NTA software utilizes the Stokes Einstein equation to determine the hydrodynamic diameters of nanoparticles, as well as the concentration of any nanoparticles present in the sample. The average of two separate thirty-second capture windows was used to estimate the concentration of nanoparticles in the sample.

High sensitivity PD-L1 assay: Atezolizumab was covalently coupled to magnetic beads (Quanterix) using the vendor’s recommended protocols. The atezolizumab-beads were diluted 1:60 in Sample Diluent (PBS,0.5% BSA, 0.05% Polysorbate 20, 0.05% Proclin 300, 0.25% CHAPS, 5 mM EDTA, 0.35 M NaCl, pH 7.4± 0.1) and a biotinylated monoclonal antibody against PD-L1 (clone 5D1 provided by our internal antibody engineering group) was diluted to 0.5 µg/mL in Sample Diluent with 1% RIPA Buffer. The Simoa HD-1 was loaded with the plate of samples, Atezolizumab beads, 150 pM streptavidin β-galactosidase and biotinylated 5D1 (non-competing with atezolizumab).

Drug tolerant PD-L1 assay: The anti-PD-L1 clone 5D1 was used as capture antibody and biotinylated 10C4 (anti-atezolizumab framework) for detection. Sample buffer is made of PBS with 10% RIPA, 10% fetal bovine serum (Thermo Fisher Scientific), 0.5% BSA, 0.25% CHAPS, 5 mM EDTA, 0.35 M NaCl, 0.05% polysorbate 20 and 0.05% ProClin-300 (Sigma-Aldrich, St. Louis, MO). Plasma or EVs lysed in RIPA buffer were mixed 1:1 with 2 µg/mL anti-PD-L1 atezolizumab on a microtiter plate and incubated on a shaker for 30 minutes. Prespecified amounts of monomeric PD-L1 (R&D Systems) were treated similarly (for standard curve). 5D1 antibody was covalently coupled to magnetic beads (Quanterix) using the vendor’s recommended protocols. The 5D1-coated beads and helper beads (in a 1:3 ratio, from Quanterix) were diluted 1:60 and the biotinylated 10C4 detection antibody was diluted to 0.5 µg/mL. Finally, the Simoa HD-1 is loaded with the plate of samples, PD-L1:5D1 antibody/helper beads, 160 pM streptavidin β-galactosidase and biotinylated 10C4.

### Tumor-Derived EVs Were Enriched Form Plasma Using Magnetic Selection With Anti-EpCAM Coated Nanoparticles

EpCAM (EPCAM or CD326) is an epithelial cell surface marker that is highly overexpressed in certain epithelial cancers (FIG. 1A, Willms et al. 2016). We analyzed TCGA RNA-seq data and found that lung adenocarcinoma and most subtypes of breast cancer display high EpCAM expression relative to their normal counterparts, with the highest expression in the basal molecular subtype, which characterizes the majority of TNBC (FIG. 1B). From our GeneLogic microarray dataset, EpCAM expression is higher in breast cancer and lung cancer as compared to adjacent normal tissue (FIG. 2). As lung and breast cancers are two of the most prevalent tumor types in the Western world, we used plasma from these patients to isolate EpCAM-positive (+) EVs using the CellSearch® platform. This technology is specific for isolating EpCAM+ particles (de Wit et al. 2015). Frozen plasma was thawed and placed into the CellTracks instrument, which would incubate the plasma with magnetic anti-EpCAM-coated beads. After several wash steps, an enriched fraction of EpCAM+ EVs would be attached to beads that remain in the sample tube. We decided to compare these EpCAM+ EVs to Total EVs isolated using DUC. A brief flowchart depicting the isolation of plasma and the subsequent extraction of these EV populations is shown in FIG. 1C.

### TEM Analysis of NSCLC, TNBC and Healthy Donor Plasma Demonstrates EVs of Various Sizes in Respective Fractions

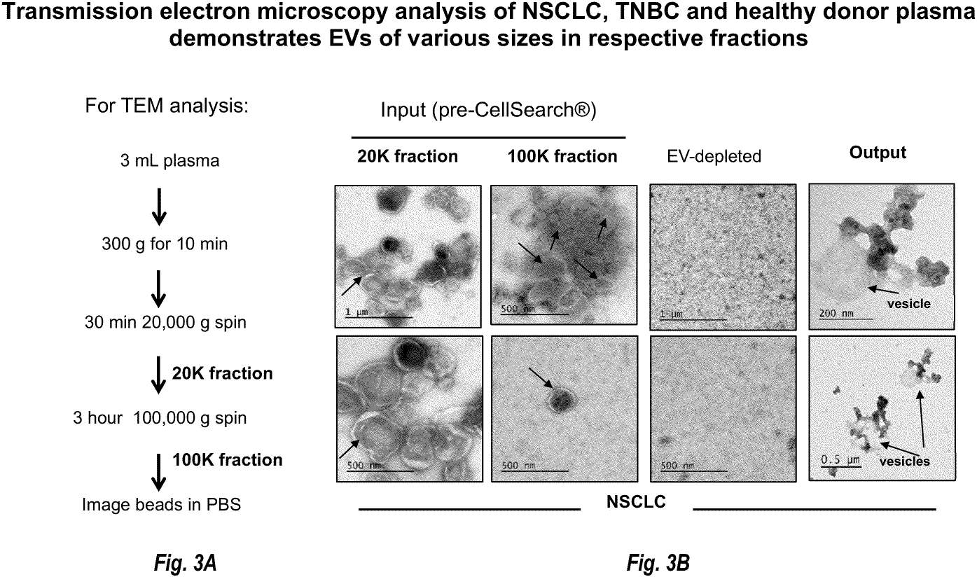

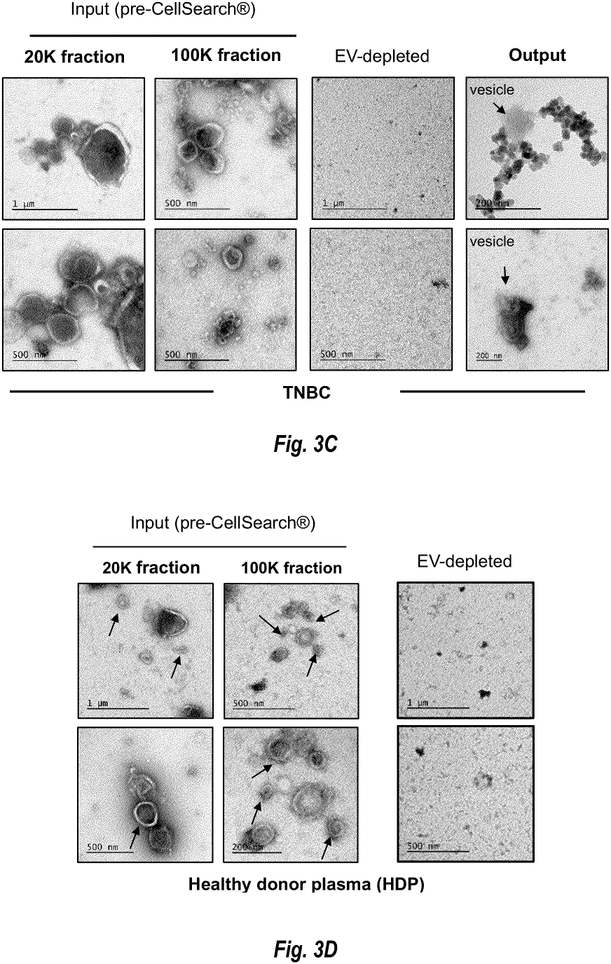

First, we characterized EV, comparing DUC (for Total EVs) and CellSearch® (for EpCAM+ EVs) EV isolation protocols. DUC was performed starting with 6 mL of healthy donor plasma (HDP) and plasma from cancer patients (FIG. 3A). For all samples, EVs were detected in the pellet after 20,000 g and then from supernatants that were subsequently spun at 100,000 g to yield an additional pellet (FIGS. 3B and 3C). As expected, no EVs were detected in the supernatant removed following the 100,000 g spin. Additionally, Nanosight tracking analysis (NTA) was performed on the 100 K fractions from NSCLC and TNBC patient plasma as well as HDP, indicating a mean particle size of around 200 nm (FIG. 4). Next, the pellet of Total EVs from DUC was resuspended in PBS. For CellSearch® enrichment, EVs were further resuspended in 3 mL of exo-free plasma and EpCAM+ EVs were isolated using a CellSearch® Profile kit. For transmission electron microscopy (TEM) analysis, the output of CellSearch® was resuspended in PBS. The output from CellSearch® showed EVs attached to anti-EpCAM beads from the NSCLC and TNBC plasma, but we did not detect any vesicles attached to anti-EpCAM beads in plasma from healthy donors. Based on these data, we will use the term “Tumor-derived EVs” interchangeably with “EpCAM+ EVs” specifically in this Examples section.

### Custom Simoa/Quanterix Assays for PD-L1 Confirm That EpCAM+ EVs Are Specific to Plasma from Cancer Patients

Next, we assessed levels of PD-L1 in Total and tumor-derived EVs. We developed two complementary PD-L1 assays on the Simoa HD-1 (Quanterix) platform - one of them with high sensitivity, while the other has a broader dynamic range and can detect PD-L1 in the presence of atezolizumab (drug tolerant PD-L1 assay). Note that these assays are different from the commercial PD-L1 assay available from Quanterix.

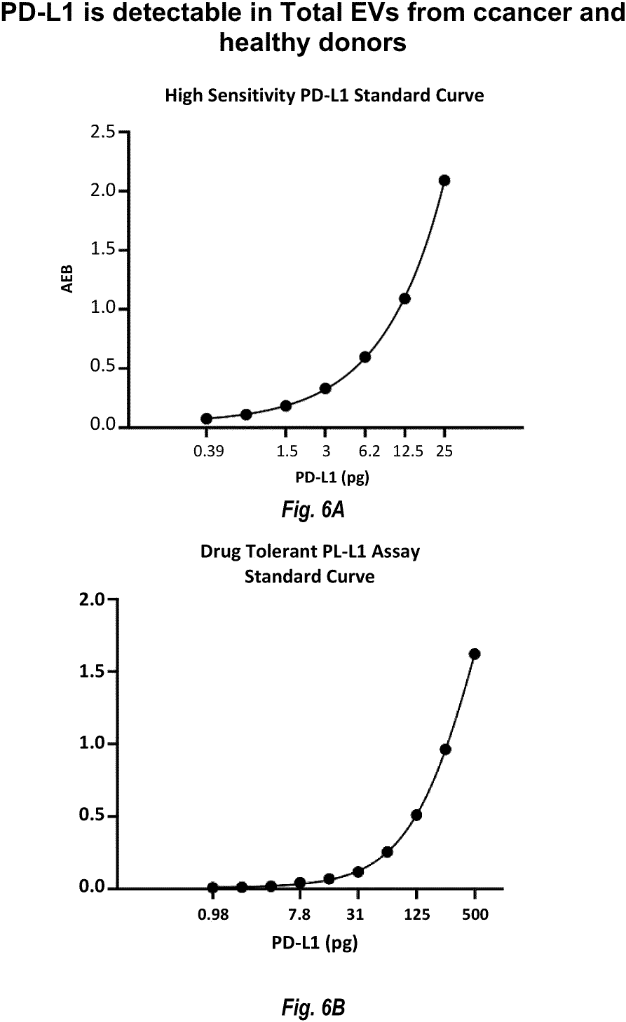

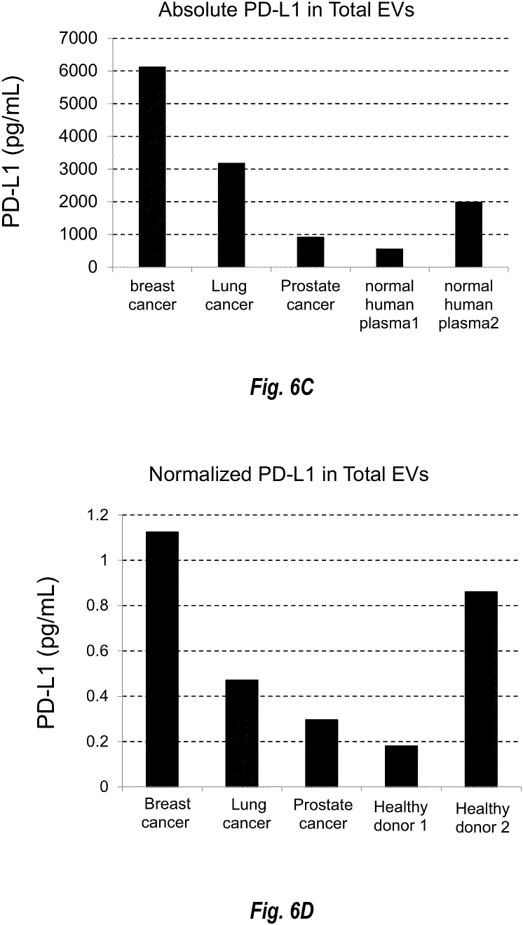

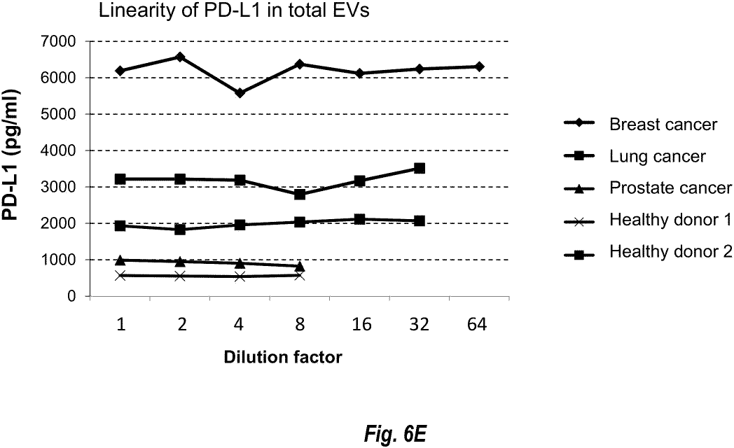

One custom PD-L1 assay uses the anti-PD-L1 antibody Atezolizumab, while the other uses the anti-PD-L1 antibody 5D1 coupled to Quanterix beads as capture (FIG. 5A). Another key difference is in the detection step - the high sensitivity assay uses biotinylated atezolizumab, while the drug tolerant assay uses two antibodies for the detection step (unlabeled atezolizumab and then a biotinylated anti-framework antibody, 10C4). As such, the dynamic range of the drug tolerant PD-L1 assay is about 1 - 500 pg/mL, while the high sensitivity PD-L1 assay can detect 0.39 - 25 pg/mL of PD-L1 (FIGS. 6A-6B).

Having both these assays in our arsenal allowed us to interrogate the level of PD-L1 in EVs from the plasma of cancer patients. We compared PD-L1 in Total EVs, which would contain PD-L1 from EVs secreted by immune cells as well as tumor cells, to EpCAM+ EVs. Again, we utilized DUC to isolate Total EVs and the CellSearch® to isolate Tumor EVs (FIG. 5C). In order to calculate the total amount of protein present, bicinchoninic acid (BCA) analysis was performed on these samples. There was a range of total protein levels in Total EVs from both cancer and healthy donor samples. However, total protein from EpCAM+ EVs was substantially higher in cancer patient plasma samples than those from healthy donors, indicating these vesicles are increased in plasma from cancer patients. For the EpCAM+ EVs, PD-L1 was observed in cancer samples only (FIG. 5D), while in Total EVs, PD-L1 was observed in samples from both cancer patients and healthy donors (FIGS. 6C and 6D). In addition, absolute and normalized (to total protein) values of PD-L1 levels from Total EVs are present in a variety of concentrations across cancer and healthy donor samples (FIGS. 6C and 6D). We tested several dilution factors for these samples, and found that our drug tolerant PD-L1 assay had acceptable linearity from 1:100 to 1:800 (< 20% variance, FIG. 6E).

In summary, our data indicates that isolating EVs using EpCAM is a viable way to obtain tumor EVs.

### Isolation of EpCAM+ EVs Is Reproducible and Specific as Determined Using EVs from Cancer Cell Lines

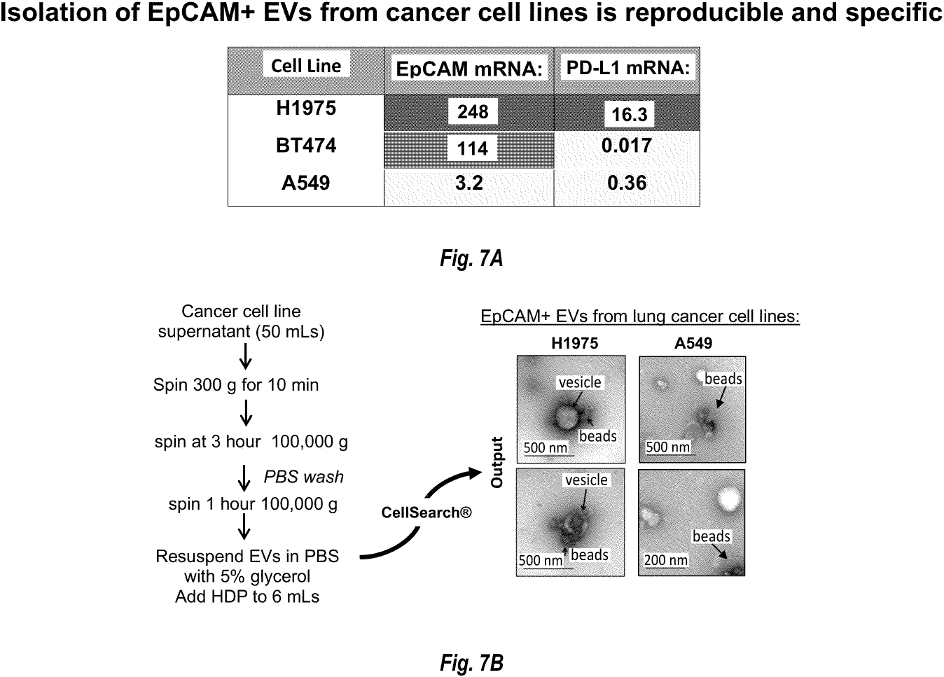

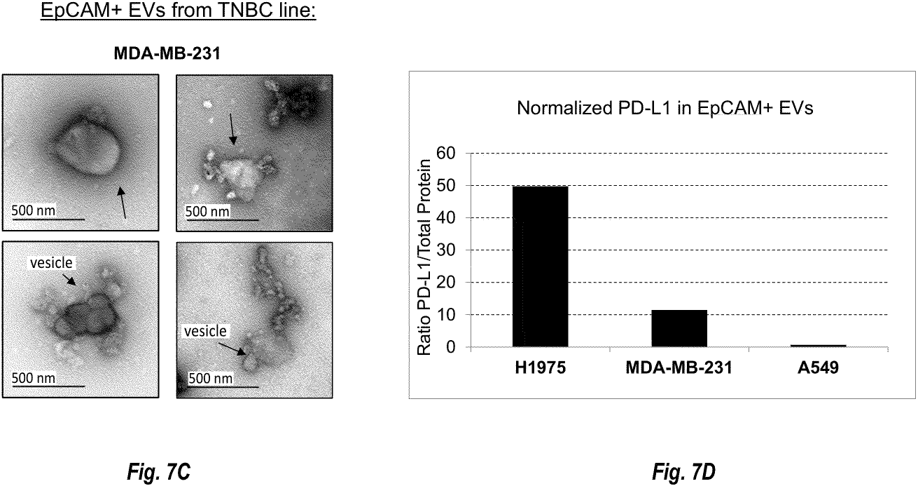

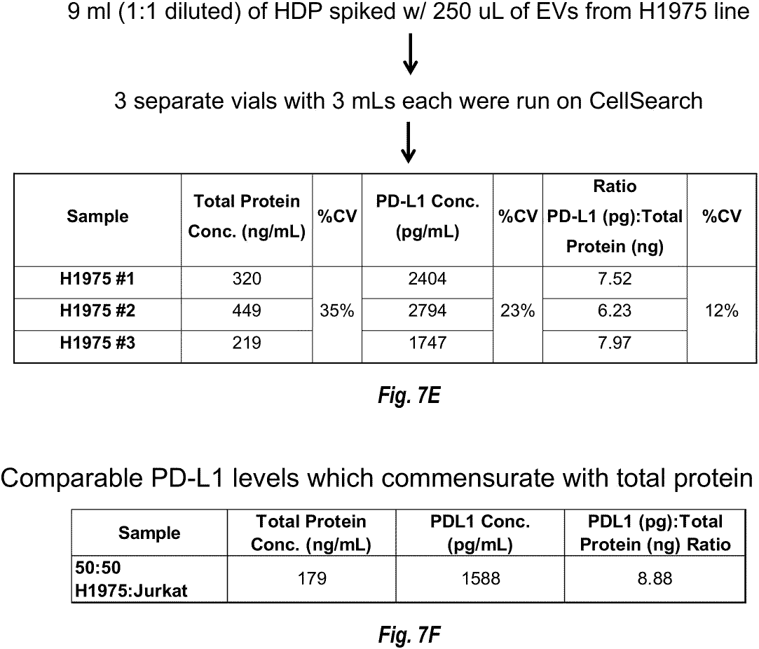

We performed further analysis of the EpCAM+ EVs from cancer cell lines to demonstrate this workflow is reproducible and specific. From Genentech’s cell line RNA-seq data, we determined that H1975 and BT474 cell lines have high RNA levels of EpCAM, as compared to A549 (NSCLC) (FIG. 7A). Additionally, high PD-L1 expression is observed in H1975 cells while extremely low expression is observed in BT474 and A549 cells. We then used the CellSearch® protocol on concentrated EVs from cell line conditioned media. To determine whether the differences in EpCAM expression levels of cells correspond to those in EVs from the respective cell lines, we examined EVs originating from cell lines with the highest and lowest EpCAM levels (FIG. 7B). Utilizing TEM negative staining, we found vesicles of various sizes (50-300 nm) attached to beads from H1975 EpCAM-high line EV concentrate. However, in the supernatant from A549, an EpCAM-low cell line, we hardly observed any vesicles and the very few microparticles that were found, were not attached to beads (FIG. 7B). We also explored a TNBC cell line. It was previously published that the TNBC line MDA-MB-231 expresses EpCAM and PD-L1 (Martowicz et al. 2012; Mittendorf et al. 2014). We performed TEM analysis of EVs isolated from these cells and indeed, we found EVs in the context of anti-EpCAM beads (FIG. 7C). To further characterize the capability to enrich for EpCAM+ EVs with the CellSearch® assay we developed, we measured PD-L1 using our two custom Simoa assays in EVs from the various cell lines. These data confirm our RNA-seq and TEM data on EpCAM enrichment (FIG. 7D).

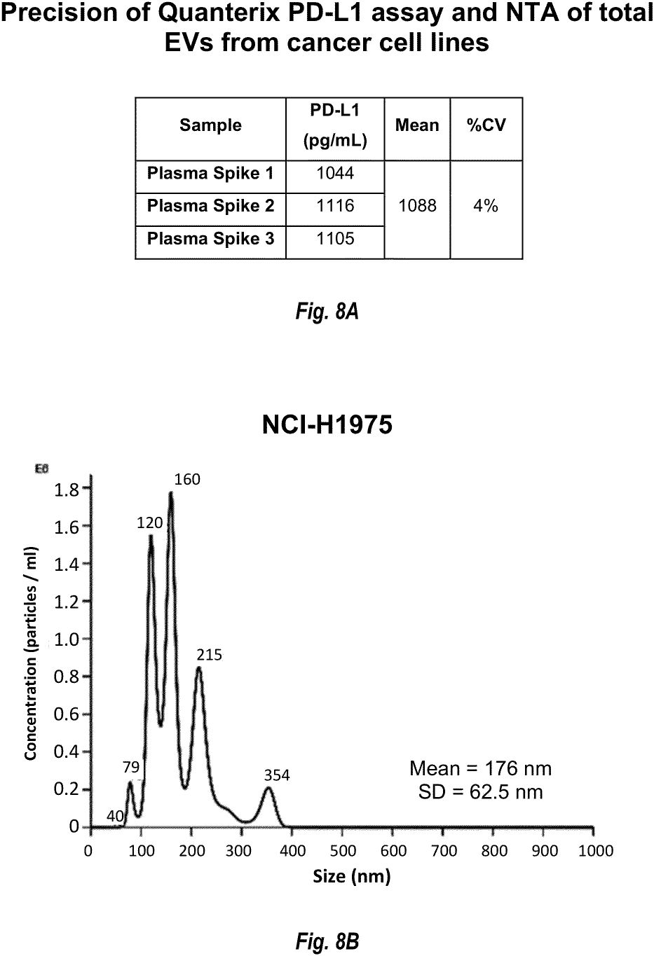

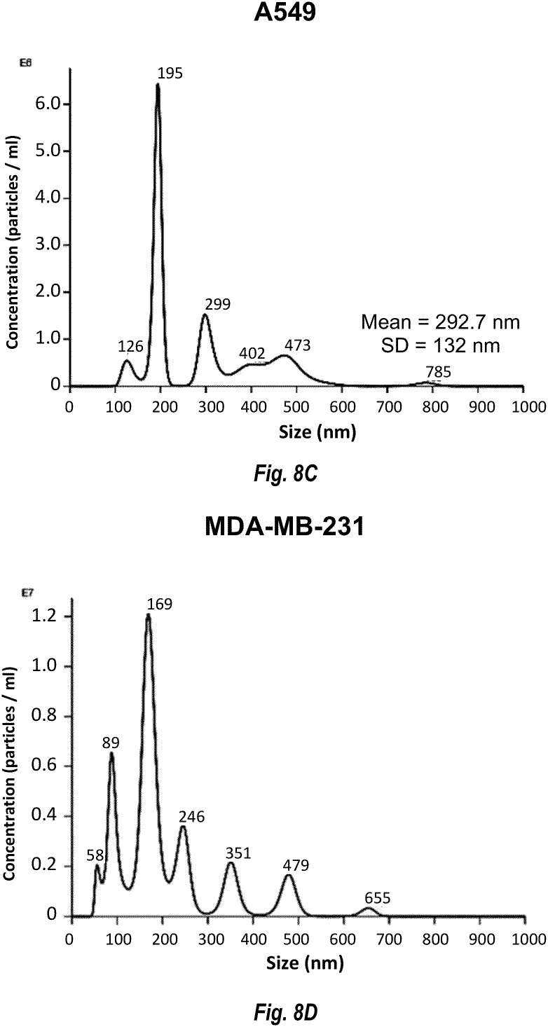

Further testing of this workflow utilized CellSearch® EpCAM+ EV enrichment and downstream testing of PD-L1 with our custom drug tolerant PD-L1 assay, due to the larger dynamic range of this assay. To assess the reproducibility of this workflow, we used three separate purifications of H1975-derived EVs spiked into healthy donor plasma. We found that %CV of PD-L1:total protein ratio is 12% (FIG. 7E). To assess specificity, plasma spiked with EpCAM-high H1975 EVs mixed into plasma spiked with EpCAM-low Jurkat EVs (50:50) was processed with CellSearch®. This led to the expected decrease in PD-L1 levels, driven by the reduced EpCAM levels of the starting material as compared with FIG. 7E (FIG. 7F). Additional NSCLC plasma samples were tested for PD-L1, which correlated well with total protein levels as measured by BCA (FIG. 8). Finally, Nanosight® analysis (NTA) was performed on DUC-isolated total EVs from these cancer cell lines. Again, the mean particle size was around 200 nm, indicating the presence of exosomes and microvesicles (FIGS. 8B and 8C). In summary, the CellSearch® method we have developed is reproducible and specific for enrichment of EpCAM+ EVs from human plasma and cancer cell lines.

### Low Starting Volumes Are Feasible for CellSearch® EpCAM Enrichment And Confirmation with the Simoa/Quanterix PD-L1 Assay of Tumor-Derived EVs

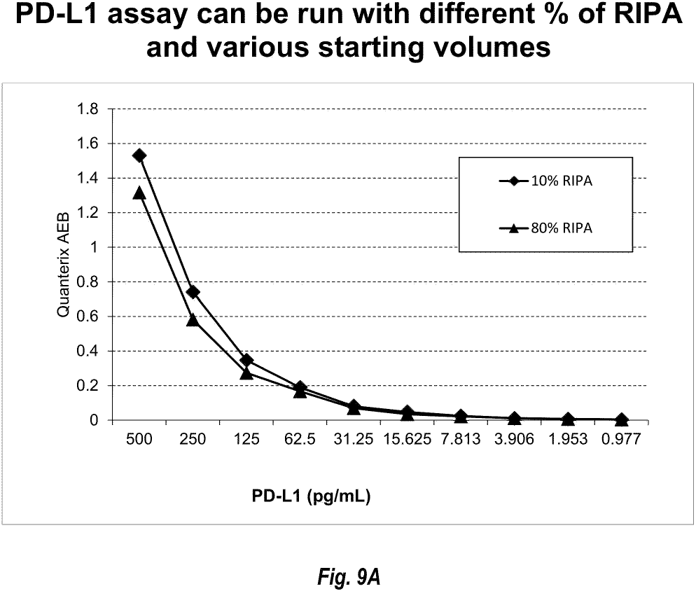

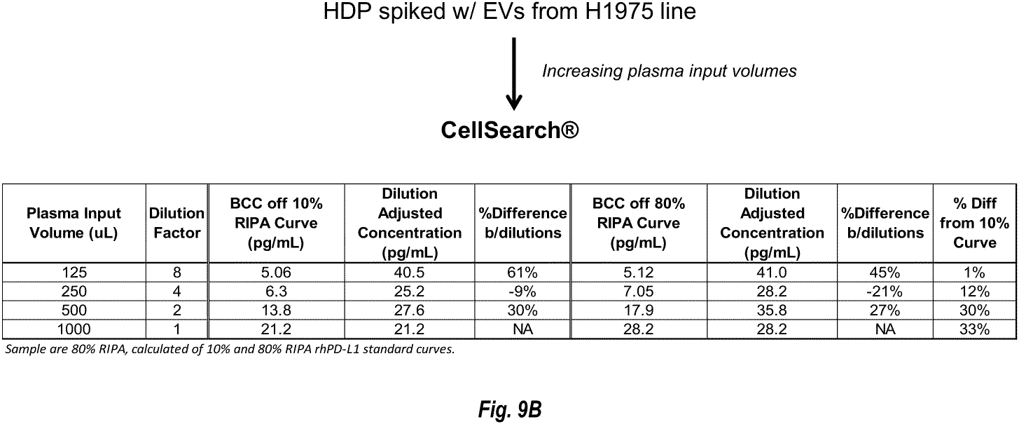

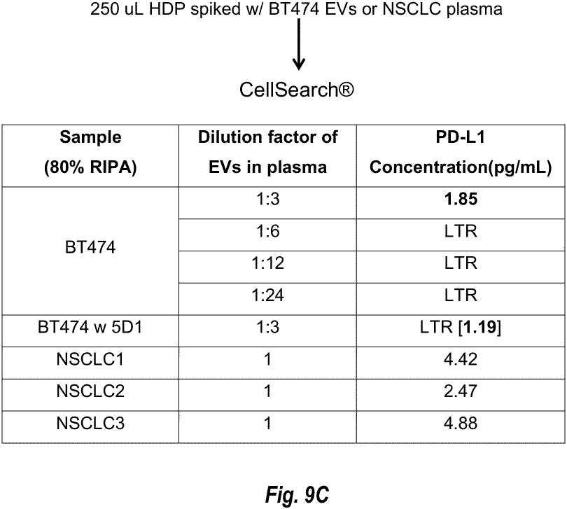

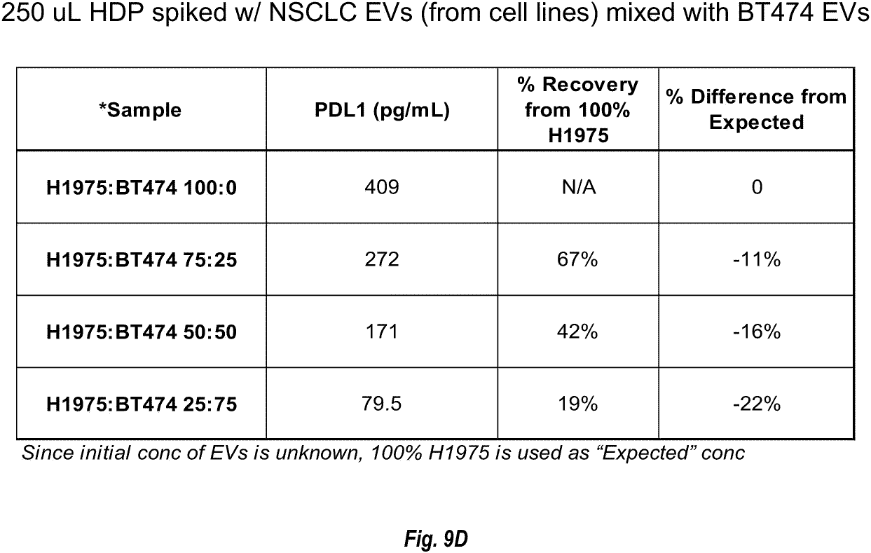

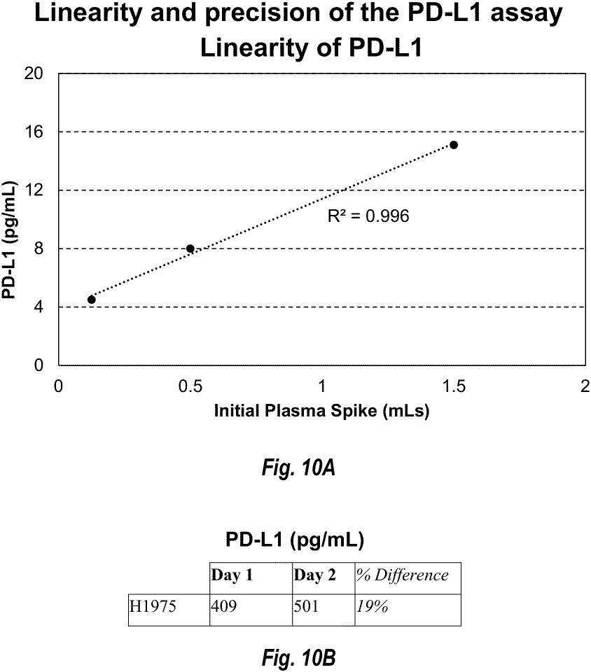

Given that plasma from clinical trials is often of limited volume, we also determined what would be the lowest possible starting volume for our tumor EV enrichment with CellSearch® followed by the custom drug tolerant PD-L1 assay. Since we wanted to run smaller volumes of concentrated samples, we needed to investigate the tolerance of the assay in the presence of various concentrations of RIPA. We initially had been using 10% RIPA. When testing as little as 250 µL of plasma containing H1975 EVs, 10% and 80% RIPA gave similar results (FIG. 9A). Then, various amounts of H1975 EVs (EpCAM-high and PD-L1-high) spiked in HDP were processed with the CellSearch® followed by PD-L1 assay workflow. A starting volume of 250 µL was the lowest that could be reliably processed using our workflow (FIG. 9B). With 250 µL of starting volume, PD-L1 levels displayed good linearity (R² = 0.996) and precision (FIGS. 10A and 10B).

Using the BT474 cell line, which is EpCAM high but PD-L1 low, we examined tumor EV PD-L1 assay sensitivity and specificity with a starting volume of 250 µL. When BT474 EVs were spiked into HDP and serially diluted, the lower threshold of PD-L1 sensitivity was 1.85 pg/mL (FIG. 9C). Any data point that quantitated below 1.85 pg/mL is denoted as “Lower Than Range” or LTR. All 3 NSCLC plasma samples quantitated above 1.85 pg/mL. In admixture experiments where an increasing fraction of BT474 EVs was mixed with H1975 EVs, we determined that our drug tolerant PD-L1 assay is specific - even with an extremely low input of 250 µL (FIG. 9D).

### DISCUSSION

In this study, we have developed a method to isolate tumor-derived EVs from NSCLC and TNBC patient plasma using CellSearch® EpCAM enrichment to enable the study of tumor-specific cargo. Notably, we show that healthy donor plasma does not harbor EpCAM+ EVs. We assessed levels of the checkpoint inhibitor PD-L1 to confirm enrichment of tumor EVs using cell culture supernatants as well as cancer patient plasma. This demonstrated the feasibility to detect relevant protein biomarkers in this EV subgroup, and would potentially be of great importance to other protein biomarkers with specific relevance to tumor cells. For instance, one group affinity-isolated prostate cancer EVs using beads coated with anti-prostate-specific membrane antigen (PSMA) antibody (Mizutani et al. 2014). A recent approach involved isolation of melanoma-derived exosomes using the marker CSPG4 (Sharma et al. 2018). Another report separated CD3+ and CD3- exosomes and found varying amounts of immunomodulatory proteins in these subsets (Theodoraki et al. 2018a). Larger studies of tumor-derived EVs from human samples are warranted.

To date, EV-mediated signaling has been implicated in numerous tumor-related processes, including chemoresistance and tumor microenvironment remodeling (Corcoran et al 2012; Ma et al 2014; Costa-Silva et al. 2015). There is some evidence that tumor-derived exosomes can impair or eradicate dendritic cells, thus facilitating immune escape (Abusamra et al. 2005; Clayton et al. 2007). Other studies have found that cancer cell line uptake of EVs influences migratory and metastatic potential (Peinado et al. 2012; Zomer et al. 2015; Steenbeek et al. 2018).

Moreover, it is clear that not only are EVs heterogeneous; there are several subsets of EVs released from cancerous tissues, each carrying different cargo with implications for tumorigenesis (Zhang et al., 2018). In our study we chose to use the marker EpCAM because it is overexpressed in various cancers, and EpCAM+ EVs from cancer cell lines were previously reported to have diverse cargo from their cancer cell-of-origin (Ji et al. 2013; Tauro et al. 2013; Zhang et al. 2016). However, there can be other subpopulations of tumor-derived EVs that are not EpCAM-positive. Since some solid tumors undergo epithelial-to-mesenchymal transition, losing some EpCAM expression, other tumor-specific enrichment markers can be assessed. Beads, such as CellSearch® beads, conjugated to other relevant markers can be manufactured for this purpose. Furthermore, this workflow can be utilized to enrich for EV subtypes from other body fluids such as serum, saliva and urine.

We recognize that EpCAM is one of many tumor-associated surface proteins commonly studied in EVs. A study of NSCLC plasma found that out of 34 cancer-associated proteins assessed, CD 171 was expressed in exosomes from 75% of the patients and it was prognostic for overall survival (Sandfeld-Paulsen et al. 2016). In another study, plasma exosomes expressing CD63 or caveolin-1 were significantly higher in melanoma patients than healthy donors (Logozzi et al. 2009). Yet another study of metastatic breast cancer patients found that HER3 and MTOR signal in EVs were associated with therapy outcome (Keup et al. 2018). In short, there may be indications where assessing one marker on EVs is informative, and other indications where multiple markers need to be utilized. Our study is a proof-of-concept of the utility of enriching for a relevant EV subtype from human plasma.

In the past decade circulating tumor cells (CTCs) have been extensively studied as a tumor-specific liquid biopsy source. However, there are several advantages for utilizing circulating tumor-derived EVs over CTCs or as complementary or alternative liquid biopsy approaches. For EVs we have used previously frozen plasma, eliminating the need to use fresh blood as in the case of CTC isolation. Also, the number of CTCs from most cancer patients, especially early-stage patients, is very low (reviewed in Kowalik et al. 2017). As EVs are often found in the 10⁸ or 10⁹ per mL range, we hypothesized assessing these will enable more robust downstream analysis.

Recently, PD-L1 in exosomes had been examined in biofluids from cancer patients. One group found that exosomal PD-L1 mRNA is predictive for anti-PD-1 response in a small cohort of NSCLC and melanoma patients (Del Re et al. 2018). Another analysis of melanoma patients found that exosomal PD-L1, but not other forms of PD-L1 in circulation, was predictive for response to pembrolizumab (Chen et al. 2018). And one more study in HNSCC patients suggested that PD-L1 in exosomes is prognostic while sPD-L1 from plasma is not (Theodoraki et al. 2018b). These and other examples indicate the value of further refining the interrogation of PD-L1 in EV subtypes, based on their cell of origin (to add to their segregation by size as proxy for their biogenesis).

To summarize, here we have developed a semi-automated enrichment method that is specific, in this Example, for tumor-derived EVs using modified CellSearch® technology. We have confirmed these data by utilizing ultra-sensitive PD-L1 custom immunoassays on cell culture supernatant and human plasma. Further, we showed that informative data can be produced from as little as 250 µL of patient plasma, and PD-L1, or another analyte within tumor-derived EVs in human plasma, may be correlated to therapeutic response. Moreover, the CellSearch® workflow developed here with EpCAM can be adapted for use with other cell surface markers, to produce informative data on other EV subsets from human plasma or other biological fluid. Other types of EVs that carry cancer-relevant cargo, can be used in liquid biopsy assays that help patients receive targeted beneficial treatment.

### REFERENCE LIST

1. Abusamra, Ashraf J. et al. “Tumor Exosomes Expressing Fas Ligand Mediate CD8+ T-Cell Apoptosis.” Blood Cells, Molecules, and Diseases, vol. 35, no. 2, 2005, pp. 169-173, doi:https://doi.org/10.1016/j.bcmd.2005.07.001.
2. Andre, Fabrice et al. “Malignant Effusions and Immunogenic Tumour-Derived Exosomes.” The Lancet, vol. 360, no. 9329, 2002, pp. 295-305, doi:https://doi.org/10.1016/50140-6736(02)09552-1.
3. Becker, Annette et al. “Extracellular Vesicles in Cancer: Cell-to-Cell Mediators of Metastasis.” Cancer Cell, vol. 30, no. 6, 2016, pp. 836-848, doi:10.1016/j.ccell.2016.10.009.
4. Bianchini, Giampaolo et al. “Triple-Negative Breast Cancer: Challenges and Opportunities of a Heterogeneous Disease.” Nature Reviews Clinical Oncology, vol. 13, 2016, p. 674, doi:10.1038/nrclinonc.2016.66.
5. Brambilla, Elisabeth et al. “Prognostic Effect of Tumor Lymphocytic Infiltration in Resectable Non-Small-Cell Lung Cancer.” Journal of Clinical Oncology, vol. 34, no. 11, 2016, pp. 1223-1230, doi:10.1200/JCO.2015.63.0970.
6. Burr, Marian L. et al. “Cmtm6 Maintains the Expression of Pd-L1 and Regulates Anti-Tumour Immunity.” Nature, vol. 549, 2017, p. 101, doi:10.1038/nature23643
7. Cohen, Steven J. et al. “Relationship of Circulating Tumor Cells to Tumor Response, Progression-Free Survival, and Overall Survival in Patients with Metastatic Colorectal Cancer.” Journal of Clinical Oncology, vol. 26, no. 19, 2008, p. 3213-3221, doi:10.1200/JCO.2007.15.8923.
8. Corcoran, Claire et al. “Docetaxel-Resistance in Prostate Cancer: Evaluating Associated Phenotypic Changes and Potential for Resistance Transfer Via Exosomes.” Plos One, vol. 7, no. 12, 2012, p. e50999, doi:10.1371/journal.pone.0050999.
9. Costa-Silva, Bruno et al. “Pancreatic Cancer Exosomes Initiate Pre-Metastatic Niche Formation in the Liver.” Nature Cell Biology, vol. 17, 2015, p. 816, doi:10.1038/ncb3169
10. https://www.nature.com/articles/ncb3169#supplementary-information.
11. Cristofanilli, Massimo et al. “Circulating Tumor Cells, Disease Progression, and Survival in Metastatic Breast Cancer.” New England Journal of Medicine, vol. 351, no. 8, 2004, pp. 781-791, doi:10.1056/NEJMoa040766.
12. de Bono, Johann S. et al. “Circulating Tumor Cells Predict Survival Benefit from Treatment in Metastatic Castration-Resistant Prostate Cancer.” Clinical Cancer Research, vol. 14, no. 19, 2008, p. 6302, http://clincancerres.aacrjournals.org/content/14/19/6302.abstract.
13. Del Re, Marzia et al. “PD-L1 mRNA Expression in Plasma-Derived Exosomes Is Associated with Response to Anti-Pd-1 Antibodies in Melanoma and NSCLC.” British Journal Of Cancer, vol. 118, 2018, p. 820, doi:10.1038/bjc.2018.9.
14. Dezutter-Dambuyant, Colette et al. “A Novel Regulation of PD-1 Ligands on Mesenchymal Stromal Cells through Mmp-Mediated Proteolytic Cleavage.” OncoImmunology, vol. 5, no. 3, 2016, p. e1091146, doi:10.1080/2162402X.2015.1091146.
15. Dong, Haidong et al. “B7-H1, a Third Member of the B7 Family, Co-Stimulates T-Cell Proliferation and Interleukin-10 Secretion.” Nature Medicine, vol. 5, 1999, p. 1365, doi:10.1038/70932.
16. Esposito, Laura et al. “Investigation of Soluble and Transmembrane CTLA-4 Isoforms in Serum and Microvesicles.” The Journal of Immunology Author Choice, vol. 193, no. 2, 2014, pp. 889-900, PMC, doi:10.4049/jimmunol.1303389.
17. Eyvazi, Shirin et al. “Antibody based EpCAM Targeted Therapy of Cancer, Review and Update”. Current Cancer Drug Targets, vol. 18, 2018, no. 9, p. 857 - 868.
18. Fehrenbacher, Louis et al. “Atezolizumab Versus Docetaxel for Patients with Previously Treated Non-Small-Cell Lung Cancer (Poplar): A Multicentre, Open-Label, Phase 2 Randomised Controlled Trial.” The Lancet, vol. 387, no. 10030, 2016, pp. 1837-1846, doi:10.1016/S0140-6736(16)00587-0.
19. Freeman, Gordon J. et al. “Engagement of the PD-1 Immunoinhibitory Receptor by a Novel B7 Family Member Leads to Negative Regulation of Lymphocyte Activation.” The Journal of Experimental Medicine, vol. 192, no. 7, 2000, p. 1027, http://jem.rupress.org/content/192/7/1027.abstract.
20. Frigola, Xavier et al. “Identification of a Soluble Form of B7-H1 That Retains Immunosuppressive Activity and Is Associated with Aggressive Renal Cell Carcinoma.” Clinical Cancer Research, vol. 17, no. 7, 2011, p. 1915, http://clincancerres.aacrjournals.org/content/17/7/1915.abstract.
21. Gastl, Günther et al. “EpCAM overexpression in breast cancer as a predictor of survival”. The Lancet, vol. 365, 2000, no. 9246, p. 1981 - 1982.
22. Gubin, Matthew M. et al. “Checkpoint Blockade Cancer Immunotherapy Targets Tumour-Specific Mutant Antigens.” Nature, vol. 515, 2014, p. 577, doi:10.1038/nature13988
23. Guibert, Nicolas et al. “PD-L1 Expression in Circulating Tumor Cells of Advanced Non-Small Cell Lung Cancer Patients Treated with Nivolumab.” Lung Cancer, vol. 120, 2018, pp. 108-112, doi:https://doi.org/10.1016/j.lungcan.2018.04.001.
24. Guo, Wei et al. “Exosomes: New Players in Cancer.” Oncology Reports, vol. 38, no. 2, 2017, pp. 665-675, PMC, doi:10.3892/or.2017.5714.
25. Herbst, Roy S. et al. “Pembrolizumab Versus Docetaxel for Previously Treated, PD-L1-Positive, Advanced Non-Small-Cell Lung Cancer (Keynote-010): A Randomised Controlled Trial.” The Lancet, vol. 387, no. 10027, 2016, pp. 1540-1550, doi:10.1016/S0140-6736(15)01281-7.
26. Hira-Miyazawa, Mayuko et al. “Regulation of Programmed-Death Ligand in the Human Head and Neck Squamous Cell Carcinoma Microenvironment Is Mediated through Matrix Metalloproteinase-Mediated Proteolytic Cleavage.” International Journal of Oncology, vol. 52, no. 2, 2018, pp. 379-388, PMC, doi:10.3892/ijo.2017.4221.
27. Huang, Jianwei et al. “Prognostic Significance of Circulating Tumor Cells in Non-Small-Cell Lung Cancer Patients: A Meta-Analysis.” PLOS ONE, vol. 8, no. 11, 2013, p. e78070, doi:10.1371/journal.pone.0078070.
28. Inal, Jameel M. et al. “Blood/Plasma Secretome and Microvesicles.” Biochimica et Biophysica Acta (BBA) - Proteins and Proteomics, vol. 1834, no. 11, 2013, pp. 2317-2325, doi:https://doi.org/10.1016/j.bbapap.2013.04.005.
29. Ji, Hong et al. “Proteome Profiling of Exosomes Derived from Human Primary and Metastatic Colorectal Cancer Cells Reveal Differential Expression of Key Metastatic Factors and Signal Transduction Components.” PROTEOMICS, vol. 13, no. 10-11, 2013, pp. 1672-1686, doi:10.1002/pmic.201200562.
30. Kazandjian, Dickran et al. “FDA Approval Summary: Nivolumab for the Treatment of Metastatic Non-Small Cell Lung Cancer with Progression on or after Platinum-Based Chemotherapy.” The Oncologist, vol. 21, no. 5, 2016, pp. 634-642, http://theoncologist.alphamedpress.org/content/21/5/634.abstract.
31. Keup, Corinna et al. “RNA Profiles of Circulating Tumor Cells and Extracellular Vesicles for Therapy Stratification of Metastatic Breast Cancer Patients.” Clinical Chemistry, vol. 64, no. 7, 2018, p. 1054, http://clinchem.aaccjnls.org/content/64/7/1054.abstract.
32. Kim, Dae-Kyum et al. “Evpedia: A Community Web Portal for Extracellular Vesicles Research.” Bioinformatics, vol. 31, no. 6, 2015, pp. 933-939, doi:10.1093/bioinformatics/btu741.
33. Kim, Youngwook et al. “Clinicopathological Implications of Epcam Expression in Adenocarcinoma of the Lung.” Anticancer Research, vol. 29, no. 5, 2009, pp. 1817-1822.
34. Konishi, Jun et al. “B7-H1 Expression on Non-Small Cell Lung Cancer Cells and Its Relationship with Tumor-Infiltrating Lymphocytes and Their PD-1 Expression.” Clinical Cancer Research, vol. 10, no. 15, 2004, p. 5094.
35. Kowalik, Artur et al. “Current Approaches for Avoiding the Limitations of Circulating Tumor Cells Detection Methods-Implications for Diagnosis and Treatment of Patients with Solid Tumors.” Translational Research, vol. 185, 2017, pp. 58-84.e15, doi:https://doi.org/10.1016/j.trsl.2017.04.002.
36. Le, Dung T. et al. “Pd-1 Blockade in Tumors with Mismatch-Repair Deficiency.” New England Journal of Medicine, vol. 372, no. 26, 2015, pp. 2509-2520, doi:10.1056/NEJMoa1500596.
37. Llosa, Nicolas J. et al. “The Vigorous Immune Microenvironment of Microsatellite Instable Colon Cancer Is Balanced by Multiple Counter-Inhibitory Checkpoints.” Cancer Discovery, vol. 5, no. 1, 2015, p. 43.
38. Logozzi, Mariantonia et al. “High Levels of Exosomes Expressing Cd63 and Caveolin-1 in Plasma of Melanoma Patients.” PLOS ONE, vol. 4, no. 4, 2009, p. e5219, doi:10.1371/journal.pone.0005219.
39. Ma, Xin et al. “Essential Role for Trpc5-Containing Extracellular Vesicles in Breast Cancer with Chemotherapeutic Resistance.” Proceedings of the National Academy of Sciences, vol. 111, no. 17, 2014, p. 6389.
40. Mathivanan, Suresh and J. Simpson Richard. “Exocarta: A Compendium of Exosomal Proteins and RNA.” Proteomics, vol. 9, no. 21, 2009, pp. 4997-5000, doi:10.1002/pmic.200900351.
41. Martowicz, Agnieszka et al. “Phenotype-dependent effects of EpCAM expression on growth and invasion of human breast cancer cell lines.” BMC Cancer, vol 501, 2012. https://doi.org/10.1186/1471-2407-12-501
42. McGranahan, Nicholas et al. “Clonal Neoantigens Elicit T Cell Immunoreactivity and Sensitivity to Immune Checkpoint Blockade.” Science, vol. 351, no. 6280, 2016, p. 1463, http://science.sciencemag.org/content/351/6280/1463.abstract.
43. Mezzadra, Riccardo et al. “Identification of Cmtm6 and Cmtm4 as Pd-L1 Protein Regulators.” Nature, vol. 549, 2017, p. 106, doi:10.1038/nature23669
44. Mittendorf, Elizabeth et al. “PD-L1 Expression in Triple Negative Breast Cancer.” Cancer Immunology Research, 2014 Apr; 2(4): 361-370. doi: 10.1158/2326-6066.CIR-13-0127
45. Mizutani, Kosuke et al. “Isolation of Prostate Cancer-Related Exosomes.” Anticancer Research, vol. 34, no. 7, 2014, pp. 3419-3423.
46. Muller, Laurent et al. “Tumor-Derived Exosomes Regulate Expression of Immune Function-Related Genes in Human T Cell Subsets.” Scientific Reports, vol. 6, 2016, p. 20254, doi:10.1038/srep20254
47. Noone AM et al (eds). SEER Cancer Statistics Review, 1975-2015, National Cancer Institute. Bethesda, MD, https://seer.cancer.gov/csr/1975_2015/, accessed April 2018.
48. Packeisen Jet al. “Detection of surface antigen 17-1A in breast and colorectal cancer.” Hybridoma, vol. 18 (1), 1999, p. 37-40.
49. Peters, Solange et al. “Phase I Trial of Atezolizumab as First-Line or Subsequent Therapy for Patients with Programmed Death-Ligand 1-Selected Advanced Non-Small-Cell Lung Cancer (Birch).” Journal of Clinical Oncology, vol. 35, no. 24, 2017, pp. 2781-2789, doi:10.1200/JCO.2016.71.9476.
50. Petrylak, D. P. et al. “Atezolizumab (MPDL3280a) Monotherapy for Patients with Metastatic Urothelial Cancer: Long-Term Outcomes from a Phase 1 Study.” JAMA Oncology, vol. 4, no. 4, 2018, pp. 537-544, doi:10.1001/jamaoncol.2017.5440.
51. Pisitkun, Trairak et al. “Identification and Proteomic Profiling of Exosomes in Human Urine.” Proceedings of the National Academy of Sciences of the United States of America, vol. 101, no. 36, 2004, p. 13368.
52. Rittmeyer, Achim et al. “Atezolizumab Versus Docetaxel in Patients with Previously Treated Non-Small-Cell Lung Cancer (OAK): A Phase 3, Open-Label, Multicentre Randomised Controlled Trial.” The Lancet, vol. 389, no. 10066, 2017, pp. 255-265, doi:10.1016/S0140-6736(16)32517-X.
53. Rossille, D. et al. “High Level of Soluble Programmed Cell Death Ligand 1 in Blood Impacts Overall Survival in Aggressive Diffuse Large B-Cell Lymphoma: Results from a French Multicenter Clinical Trial.” Leukemia, vol. 28, 2014, p. 2367, doi:10.1038/leu.2014.137
54. Sandfeld-Paulsen, B. et al. “Exosomal Proteins as Prognostic Biomarkers in Non-Small Cell Lung Cancer.” Molecular Oncology, vol. 10, no. 10, 2016, pp. 1595-1602, doi:https://doi.org/10.1016/j.molonc.2016.10.003.
55. Schmid, Peter et al. “Atezolizumab and Nab-Paclitaxel in Advanced Triple-Negative Breast Cancer.” New England Journal of Medicine, vol. 379, no. 22, 2018, pp. 2108-2121, doi:10.1056/NEJMoa1809615.
56. Sharma, Priyanka et al. “Immunoaffinity-based isolation of melanoma cell-derived exosomes from plasma of patients with melanoma”. Journal of Extracellular Vesicles, vol. 7, 2018, no. 1, 1435138. doi: 10.1080/20013078.2018.
57. Socinski, Mark A. et al. “Atezolizumab for First-Line Treatment of Metastatic Nonsquamous Nsclc.” New England Journal of Medicine, vol. 378, no. 24, 2018, pp. 2288-2301, doi:10.1056/NEJMoa1716948.
58. Sul, Joohee et al. “FDA Approval Summary: Pembrolizumab for the Treatment of Patients with Metastatic Non-Small Cell Lung Cancer Whose Tumors Express Programmed Death-Ligand 1.” The Oncologist, vol. 21, no. 5, 2016, pp. 643-650.
59. Sun, Chong et al. “Regulation and Function of the Pd-L1 Checkpoint.” Immunity, vol. 48, no. 3, 2018, pp. 434-452, doi:https://doi.org/10.1016/j.immuni.2018.03.014.
60. Takahashi, Naoki et al. “Serum Levels of Soluble Programmed Cell Death Ligand 1 as a Prognostic Factor on the First-Line Treatment of Metastatic or Recurrent Gastric Cancer.” Journal of Cancer Research and Clinical Oncology, vol. 142, no. 8, 2016, pp. 1727-1738, doi:10.1007/s00432-016-2184-6.
61. Tamura, Hideto et al. “B7-H1 Costimulation Preferentially Enhances Cd28-Independent T-Helper Cell Function.” Blood, vol. 97, no. 6, 2001, p. 1809.
62. Tauro, Bow J. et al. “Two Distinct Populations of Exosomes Are Released from Lim1863 Colon Carcinoma Cell-Derived Organoids.” Molecular & Cellular Proteomics, vol. 12, no. 3, 2013, p. 587, http://www.mcponline.org/content/12/3/587.abstract.
63. Theodoraki, Marie-Nicole et al. “Separation of plasma-derived exosomes into CD3(+) and CD3(-) fractions allows for association of immune cell and tumour cell markers with disease activity in HNSCC patients”. Clinical & Experimental Immunology, 2018, vol. 194, no. 1.
64. Theodoraki, Marie-Nicole et al. “Clinical Significance of PD-L1 Exosomes in Plasma of Head and Neck Cancer Patients.” Clinical Cancer Research, vol. 24, no. 4, 2018, p. 896, http://clincancerres.aacrjournals.org/content/24/4/896.abstract.
65. Thompson, R. Houston et al. “Costimulatory B7-H1 in Renal Cell Carcinoma Patients: Indicator of Tumor Aggressiveness and Potential Therapeutic Target.” Proceedings of the National Academy of Sciences of the United States of America, vol. 101, no. 49, 2004, p. 17174.
66. van der Gun, Bernardina T. F. et al. “Epcam in Carcinogenesis: The Good, the Bad or the Ugly.” Carcinogenesis, vol. 31, no. 11, 2010, pp. 1913-1921, doi:10.1093/carcin/bgq187.
67. Waldenstrom, Anders et al. “Cardiomyocyte Microvesicles Contain DNA/Rna and Convey Biological Messages to Target Cells.” PLOS ONE, vol. 7, no. 4, 2012, p. e34653, doi:10.1371/journal.pone.0034653.
68. Willms, A. et al. “Tumour-Associated Circulating Microparticles: A Novel Liquid Biopsy Tool for Screening and Therapy Monitoring of Colorectal Carcinoma and Other Epithelial Neoplasia.” no. 1949-2553 (Electronic).
69. Wit, Sanne de et al. “The Detection of Epcam+ and Epcam- Circulating Tumor Cells.” Scientific Reports, vol. 5, 2015, p. 12270, doi:10.1038/srep12270
70. Yamazaki, Tomohide et al. “Expression of Programmed Death 1 Ligands by Murine T Cells and Apc.” The Journal of Immunology, vol. 169, no. 10, 2002, p. 5538.
71. Yu, Mamie, et al. A resource for cell line authentication, annotation and quality control. Nature 2015; vol. 520, no. 7547, p. 307-11.
72. Zhang, Wei et al. “Characterization of Exosomes Derived from Ovarian Cancer Cells and Normal Ovarian Epithelial Cells by Nanoparticle Tracking Analysis.” Tumor Biology, vol. 37, no. 3, 2016, pp. 4213-4221, doi:10.1007/s13277-015-4105-8.
73. Zhu, Xinxin and Jinghe Lang. “Soluble PD-1 and PD-L1: Predictive and Prognostic Significance in Cancer.” Oncotarget, vol. 8, no. 57, 2017, p. 97671-97682, PMC, doi:10.18632/oncotarget.18311.
74. Zomer, Anoek et al. “In vivo Imaging Reveals Extracellular Vesicle-Mediated Phenocopying of Metastatic Behavior.” Cell, vol. 161, no. 5, 2015, pp. 1046-1057, doi:https://doi.org/10.1016/j.cell.2015.04.042.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference. In addition, the entire contents of U.S. Provisional Application No. 63/011,583 filed Apr. 17, 2020, and to which priority is claimed, are also incorporated herein by reference.

1. A process of enriching cell-type specific extracellular vesicles (EVs) in a biological fluid sample from a subject, comprising:
a. providing a biological fluid sample from the subject;
b. contacting the sample with antibodies specific for a cell surface marker for one or more cell types, optionally wherein the antibodies are attached to a matrix; and
c. isolating antibody-bound EVs from unbound EVs in the sample.

2. The process of claim 1, wherein the sample comprises a volume from 100 µl to 7 mL, from 200 µl to 6.5 mL, from 250 µl to 6.5 mL, from 200 µl to 6 mL, from 250 µl to 6 mL, from 200 µl to 5 mL, from 200 µl to 4 mL, from 200 µl to 3 mL, from 200 µl to 2 mL, from 100 µl to 1 mL, from 150 µl to 1 mL, from 200 µl to 1 mL, from 250 µl to 1 mL, from 200 µl to 500 µl, from 200 µl to 500 µl, from 200 µl to 400 µl, from 250 µl to 500 µl, or from 250 µl to 400 µl.

3. (canceled)

4. The process of claim 1, wherein the cell surface marker is an epithelial cell surface marker.

5. The process of claim 1, wherein the cell surface marker is:
a. an epithelial cell surface marker, optionally wherein the epithelial cell surface marker is EpCAM, EGFR, PSMA, GSP64, CD3, CD49b, CD87, CD95, E-Cadherin CA9, CA12, N-cadherin, OB-cadherin, cadherin-11, a cytokeratin or epithelial membrane antigen (EMA);
b. a lung tissue marker, optionally wherein the lung tissue marker is EpCAM, CA9, CA12, DSG3, FAT2, GPR87, KISS1R, LYPD3, SLC7A11, TMPRSS4, CD133, prominin-1, AC133, programmed death-1 receptor (PD1) or FAS;
c. a breast tissue marker, optionally wherein the breast tissue marker is E-cadherin, epithelial membrane antigen (EMA), human epidermal growth factor receptor type 2 (Her2/neu), αvβ6 integrin, EpCAM, carcinoembryonic antigen (CEA), folate receptor-alpha (FR-α), urokinase-type plasminogen activator receptor (uPAR) or placental-specific protein 1 (PLAC1);
d. a liver cell marker, optionally wherein the liver cell marker is CD133, Prominin-1, CD44, EpCAM, delta-like 1 non-canonical Notch ligand 1 (DLK1), ALDH, CD13, CD90, CD24, OV6, ICAM-1, CD34, C-kit, α2δ1, K19, LGR5, GPC3, Annexin A2, CD15, ABC transporters, Nope, DCLK1, ASGPR, CK or CD47;
e. a glioblastoma biomarker, optionally wherein the glioblastoma biomarker is fibronectin, CD63, HSP70, Annexin A2, CD9, CD81, CD44, GRP78, CD133, CD15, a sialoglycoprotein, SLC1A3, PTPRZ1, GPR56, CLU or ALD1A3;
f. a urinary tract cell marker, optionally wherein the urinary tract cell marker is a tetraspanin, CD9, CD81, LAMP-1, CD10, CD24, CD44 or CD63; or
g. an amyloid marker, optionally wherein the amyloid marker is beta-amyloid precursor protein (β-APP), presenilin protein PS-1, or proinsulin protein PS-2.

6-7. (canceled)

8. The process of claim 1, wherein the sample comprises serum or plasma.

9. (canceled)

10. The process of claim 1, wherein the subject is a cancer subject, a suspected cancer subject, an infectious disease subject, or a suspected infectious disease subject.

11-16. (canceled)

17. The process of claim 1, further comprising isolating total extracellular vesicles (EVs) from the sample by membrane capture or differential ultracentrifugation, determining total protein levels in the isolated total extracellular vesicles, and optionally comparing the total protein levels from the total EVs to levels of cell surface marker in the EVs.

18. The process of claim 1, wherein isolation of antibody bound EVs from unbound EVs is through attachment to the matrix.

19. (canceled)

20. The process of claim 1, further comprising contacting the antibody bound EVs with a detection agent specific for a second biomarker.

21-23. (canceled)

24. The process of claim 20, wherein
a. the subject has cancer or is suspected of having cancer, and
b. where the second biomarker is
i. a polypeptide expressed or overexpressed in tumor cells,
ii. a cytokine, cytokine receptor, or an immune checkpoint regulator, and /or
iii. one or more of PD-L1, SCF, IL-3, GM-CSF, G-CSF, M-CSF, TNF-alpha, IL-2, IL-5, TMB, CTLA-4, ICOS, 4-1BB (CD137), PD-1, CTLA-4, LAG-3, Tim-3, CD39, IFN-β, IFN-γ, IL-2, IL-10, TGF-B, CCR10, CXCR4, CCR7, sMICA, IL-8, IDO1, GBP1, class II MHC molecules, CXCL9, CXCL10 (IP-10), CXCL11, IL-6, CCL4, CCL5, IFNGR1, IFNGR2, JAK2, IRF1, IFIT1, IFIT2, MTAP, miR3, SOCA1, PIAS4, GZMA, GZMB, PRF1, HLA-DQA1, HLA-DRB1, IFNG, STAT1, ICAM1-5, VCAM-1, JAK1, JAK2, CCR5, HLA-DRA, CXCR6, TIGIT, CD27, CD274, PDCD1LG2 (PD-L2), LAG3, NKG7, PSMB10, CMKLR1, CD8A, CD8B, HLA-DB1, HLA-E, CD276, CD3D, CD3E, CD3G, CD247, ZAP70, CD2, CD28, ICOS, IL12Rb1, GZMM, FLTSLG, IL-15, Eotazin, GRO-1, VEGF, HGF, AFP, BCR-ABL, BRCA1/BRCA2, B-Raf V600E, CA-125, CA19-9, CEA, EGFR, HER-2/neu, KIT, PSA, S100, KRAS, UGT1A1 or CD20.

25. (canceled)

26. The process of claim 1, wherein the process has one or more of the following characteristics:
the sample is not subjected to chromatography either before or after contacting the sample with the antibodies;
the sample is not treated before contacting the sample with the antibodies other than optionally to remove particles above 1 µm, above 2 µm, above 5 µm, above 10 µm, or above 20 µm in diameter and/or to remove cellular bodies and debris larger than EVs;
the EVs are not subjected to chromatography either before or after contacting the sample with the antibodies;
the antibodies are attached to a matrix, and the matrix is not a filter, an ion-exchange medium, or a membrane; or
the antibodies are attached to a matrix and the matrix is not charged.

27. The process of claim 1, wherein isolating the cell-type specific EVs from the sample consists essentially of: contacting the sample with antibodies specific for the cell surface marker for the one or more cell types, optionally wherein the antibodies are attached to a matrix; and isolating the antibody-bound EVs from unbound EVs in the sample.

28. A process of enriching cell-type specific extracellular vesicles (EVs) from a subject, comprising:
a. providing a biological fluid sample from the subject;
b. contacting the sample with antibodies specific for a cell surface marker, optionally wherein the antibodies are attached to a matrix;
c. isolating antibody-bound EVs from unbound EVs in the sample;
d. optionally resuspending the antibody-bound EVs in a lysis buffer; and
e. contacting the EVs with a detection agent specific for a second biomarker; optionally wherein
the cell surface marker is:
i. an epithelial cell surface marker, optionally wherein EpCAM, EGFR, PSMA, GSP64, CD3, CD49b, CD87, CD95, E-Cadherin CA9, CA12, N-cadherin, OB-cadherin, cadherin-11, a cytokeratin or epithelial membrane antigen (EMA);
ii. a lung tissue marker, optionally wherein EpCAM, CA9, CA12, DSG3, FAT2, GPR87, KISS1R, LYPD3, SLC7A11, TMPRSS4, CD133, prominin-1, AC133, programmed death-1 receptor (PD1) or FAS;
iii. breast tissue marker, optionally wherein E-cadherin, epithelial membrane antigen (EMA), human epidermal growth factor receptor type 2 (Her2/neu), αvβ6 integrin, EpCAM, carcinoembryonic antigen (CEA), folate receptor-alpha (FR-α), urokinase-type plasminogen activator receptor (uPAR) or placental-specific protein 1 (PLAC1);
iv. a liver cell marker, optionally wherein CD133, Prominin-1, CD44, EpCAM, delta-like 1 non-canonical Notch ligand 1 (DLK1), ALDH, CD13, CD90, CD24, OV6, ICAM-1, CD34, C-kit, α2δ1, K19, LGR5, GPC3, Annexin A2, CD15, ABC transporters, Nope, DCLK1, ASGPR, CK or CD47;
v. a glioblastoma biomarker, optionally wherein fibronectin, CD63, HSP70, Annexin A2, CD9, CD81, CD44, GRP78, CD133, CD15, a sialoglycoprotein, SLC1A3, PTPRZ1, GPR56, CLU or ALD1A3;
vi. a urinary tract cell marker, optionally wherein a tetraspanin, CD9, CD81, LAMP-1, CD10, CD24, CD44 or CD63; or
vii. an amyloid marker optionally wherein beta-amyloid precursor protein (β-APP), presenilin protein PS-1, or proinsulin protein PS-2.

29-46. (canceled)

47. The process of claim 28, further comprising isolating total extracellular vesicles (EVs) from the sample by membrane capture or differential ultracentrifugation, determining total protein levels in the isolated total extracellular vesicles, and optionally comparing the total protein levels from the total EVs to levels of cell surface marker in the EVs.

48. The process of claim 28, wherein isolation of antibody bound EVs from unbound EVs is through attachment to the matrix.

49. (canceled)

50. The process of claim 28, wherein the process has one or more of the following characteristics:
the sample is not subjected to chromatography either before or after contacting the sample with the antibodies;
the sample is not treated before contacting the sample with the antibodies other than optionally to remove particles above 1 µm, above 2 µm, above 5 µm, above 10 µm, or above 20 µm in diameter from the sample and/or to remove cellular bodies and debris larger than EVs;
the EVs are not subjected to chromatography either before or after contacting the sample with the antibodies;
the antibodies are attached to a matrix, and the matrix is not a filter, an ion-exchange medium, or a membrane; or
the antibodies are attached to a matrix and the matrix is not charged.

51. The process of claim 28, wherein isolating the cell-type specific EVs from the sample consists essentially of: contacting the sample with antibodies specific for the cell surface marker for the one or more cell types, optionally wherein the antibodies are attached to a matrix; isolating the antibody-bound EVs from unbound EVs in the sample, resuspending the antibody-bound EVs in a lysis buffer, and contacting the EVs with a detection agent specific for a second biomarker.

52-53. (canceled)

54. The process of claim 28, wherein the subject has cancer or is suspected of having cancer, and wherein the second biomarker is a polypeptide expressed or overexpressed in tumor cells, and/or wherein the second biomarker is a cytokine, cytokine receptor, or an immune checkpoint regulator chosen from one or more of PD-L1, SCF, IL-3, GM-CSF, G-CSF, M-CSF, TNF-alpha, IL-2, IL-5, TMB, CTLA-4, ICOS, 4-1BB (CD137), PD-1, CTLA-4, LAG-3, Tim-3, CD39, IFN-β, IFN-γ, IL-2, IL-10, TGF-β, CCR10, CXCR4, CCR7, sMICA, IL-8, IDO1, GBP1, class II MHC molecules, CXCL9, CXCL10 (IP-10), CXCL11, IL-6, CCL4, CCL5, IFNGR1, IFNGR2, JAK2, IRF1, IFIT1, IFIT2, MTAP, miR3, SOCA1, PIAS4, GZMA, GZMB, PRF1, HLA-DQA1, HLA-DRB1, IFNG, STAT1, ICAM1-5, VCAM-1, JAK1, JAK2, CCR5, HLA-DRA, CXCR6, TIGIT, CD27, CD274, PDCD1LG2 (PD-L2), LAG3, NKG7, PSMB10, CMKLR1, CD8A, CD8B, HLA-DB1, HLA-E, CD276, CD3D, CD3E, CD3G, CD247, ZAP70, CD2, CD28, ICOS, IL12Rb1, GZMM, FLTSLG, IL-15, Eotazin, GRO-1, VEGF, HGF, AFP, BCR-ABL, BRCA1/BRCA2, B-Raf V600E, CA-125, CA19-9, CEA, EGFR, HER-2/neu, KIT, PSA, S100, KRAS, UGT1A1 or CD20.

55-57. (canceled)

58. A process of identifying tumor-derived extracellular vesicles (EVs) in a subject, comprising:
a. providing a plasma sample from the subject, optionally wherein the sample is from 100 µl to 1 mL, 200 µl to 1 mL, from 250 µl to 1 mL, from 100 µl to 500 µl, from 200 µl to 500 µl, from 200 µl to 400 µl, from 250 µl to 500 µl, or from 250 µl to 400 µl;
b. contacting the plasma sample with antibodies specific for EpCAM, optionally wherein the antibodies are attached to a matrix;
c. isolating EpCAM antibody-bound EVs from unbound EVs in the sample;
d. optionally resuspending the EpCAM antibody-bound EVs in a lysis buffer;
and
e. contacting the EpCAM antibody-bound EVs with an antibody specific for PD-L1 and optionally determining the level of PD-L1 in the EpCAM antibody-bound EVs; optionally wherein the subject is a human having or suspected of having breast cancer, triple negative breast cancer, lung cancer, NSCLC, SCLC, liver cancer, urinary tract cancer, bladder cancer, brain cancer, or glioblastoma.

59. A process of determining treatment for a subject with cancer, comprising:
a. providing a plasma sample from the subject, optionally wherein the sample is 100 µl to 1 mL, from 200 µl to 1 mL, from 250 µl to 1 mL, from 100 µl to 500 µl, from 200 µl to 500 µl, from 200 µl to 400 µl, from 250 µl to 500 µl, or from 250 µl to 400 µl;
b. contacting the plasma sample with antibodies specific for EpCAM, optionally wherein the antibodies are attached to a matrix;
c. isolating antibody-bound extracellular vesicles (EVs) from unbound EVs in the sample;
d. optionally resuspending the EpCAM antibody-bound EVs in a lysis buffer;
e. contacting the EVs with an antibody specific for PD-L1;
f. determining the level of PD-L1 in the EpCAM antibody-bound EVs;
g. determining whether the subject should receive treatment with an immune checkpoint inhibitor (e.g. a PD-1 or PD-L1 inhibitor) based on the determined level of PD-L1 in the EpCAM antibody-bound EVs, wherein increases in PD-L1 levels correlate with need for treatment with the inhibitor; optionally wherein the treatment is chosen from:
i. a PD-L1 inhibitor, optionally wherein the PD-L1 inhibitor is atezolimumab, durvalumab, avelumab, envafolimab, BMS-936559, CK-301, CS-1001, SHR-1316, CBT-502, or BGB-A333;
ii. a PD-1 inhibitor, optionally wherein the PD-1 inhibitor is nivolumab, pembrolizumab, cemiplimab, spartalizumab, camrelizumab, sintilimab, tislelizumab, toripalimab, AMP-224, or AMP-514; or
iii. a CTLA-4 inhibitor, optionally wherein the CTLA-4 inhibitor is ipilimumab.

60-65. (canceled)

66. A kit for enriching or identifying cell-type specific extracellular vesicles (EVs) in a biological fluid sample from a subject, the kit comprising antibodies specific for a cell surface marker attached to a matrix, and optionally further comprising: (a) one or more detection reagents for detection of a second biomarker, (b) one or more buffers for resuspending antibody-bound EVs and/or for detection of a second biomarker, and (c) instructions for use in enriching EVs from a biological fluid sample of a subject; optionally wherein the cell surface marker is:
i. an epithelial cell surface marker, optionally wherein the epithelial cell surface marker is EpCAM, EGFR, PSMA, GSP64, CD3, CD49b, CD87, CD95, E-Cadherin CA9, CA12, N-cadherin, OB-cadherin, cadherin-11, a cytokeratin or epithelial membrane antigen (EMA);
ii. a lung tissue marker, optionally wherein the lung tissue marker is EpCAM, CA9, CA12, DSG3, FAT2, GPR87, KISS1R, LYPD3, SLC7A11, TMPRSS4, CD133, prominin-1, AC133, programmed death-1 receptor (PD1) or FAS;
iii. a breast tissue marker, optionally wherein the breast tissue marker is E-cadherin, epithelial membrane antigen (EMA), human epidermal growth factor receptor type 2 (Her2/neu), αvβ6 integrin, EpCAM, carcinoembryonic antigen (CEA), folate receptor-alpha (FR-α), urokinase-type plasminogen activator receptor (uPAR) or placental-specific protein 1 (PLAC1);
iv. a liver cell marker, optionally wherein the liver cell marker is CD133, Prominin-1, CD44, EpCAM, delta-like 1 non-canonical Notch ligand 1 (DLK1), ALDH, CD13, CD90, CD24, OV6, ICAM-1, CD34, C-kit, α2δ1, K19, LGR5, GPC3, Annexin A2, CD15, ABC transporters, Nope, DCLK1, ASGPR, CK or CD47;
v. a glioblastoma biomarker, optionally wherein the glioblastoma biomarker is fibronectin, CD63, HSP70, Annexin A2, CD9, CD81, CD44, GRP78, CD133, CD15, a sialoglycoprotein, SLC1A3, PTPRZ1, GPR56, CLU or ALD1A3;
vi. a urinary tract cell marker, optionally wherein the urinary tract cell marker is a tetraspanin, CD9, CD81, LAMP-1, CD10, CD24, CD44 or CD63; or
vii. an amyloid marker, optionally wherein the amyloid marker is beta-amyloid precursor protein (β-APP), presenilin protein PS-1, or proinsulin protein PS-2.

67-71. (canceled)
